(19) United States
(12) Patent Application Publication
(10) Pub. No.: US 2017/0211104 A1
(43) Pub. Date: Jul. 27, 2017

(54) BIOSYNTHETIC PRODUCTION OF ACETAMINOPHEN, P-AMINOPHENOL, AND P-AMINOBENZOIC ACID

(71) Applicant: 20n Labs, Inc., Washington, DC (US)

(72) Inventors: John Christopher Anderson, Berkeley, CA (US); Saurabh Srivastava, San Francisco, CA (US); Mark T. Daly, Oakland, CA (US); Patrick Poon, San Francisco, CA (US); Timothy Revak, Oakland, CA (US)

(21) Appl. No.: 15/408,319

(22) Filed: Jan. 17, 2017

Related U.S. Application Data
(60) Provisional application No. 62/281,622, filed on Jan. 21, 2016.

(51) Int. Cl. C12P 13/00

(52) U.S. Cl. CPC C12P 13/001; C12Y 206/01085; C12Y 401/03038; C12Y 114/13027; C12Y 203/01118

(57) ABSTRACT

The present disclosure provides compositions and methods for the biosynthetic production of acetaminophen, p-aminophenol, and p-aminobenzoic acid and the purification of biologically derived acetaminophen.

### CROSS REFERENCE TO RELATED APPLICATIONS

This application claims priority to, and benefit of U.S. Provisional Application No. 62/281,622 filed Jan. 21, 2016, the contents of which are incorporated herein by reference in its entirety.

### INCORPORATION-BY-REFERENCE OF SEQUENCE LISTING

The contents of the text file named “NLAB_003_01US_ST25.txt” submitted electronically herewith which was created on Jan. 5, 2017 and is 77 KB in size, are incorporated herein by reference in their entirety.

### FIELD OF THE INVENTION

The present disclosure relates compositions and methods for the biosynthetic production of medicinal supplements such as acetaminophen and intermediates including p-aminophenol, poly(p-aminophenol), and p-aminobenzoic acid (PABA). In particular, the disclosure features recombinant microorganisms comprising an engineered acetaminophen biosynthesis pathway. The disclosure features processes to isolate and purify biologically derived acetaminophen,

### BACKGROUND OF THE INVENTION

Acetaminophen is a popular analgesic sold in tablet form under various trade names including Tylenol. It is considered by the WHO as an “essential medicine” that should “be available at all times in adequate amounts and in appropriate dosage forms, at a price the community can afford.” it is currently produced via various multi-step chemical routes in large, dedicated factories.

There are many synthetic chemistry routes to acetaminophen, most of which begin with phenol derived from benzene (both carcinogens). Examples include the original Boots method which involves nitration of phenol with sulfuric acid and sodium nitrate giving a mixture of two isomers, from which the desired 4-nitrophenol is separated by steam distillation. The nitro group is then reduced to amine giving 4-aminophenol which is acetylated with acetic anhydride to produce acetaminophen. A “greener” method from Hoeschst-Celanese involving direct acetylation of phenol with acetic anhydride catalyzed by hydrofluoric acid. There is currently no synthetic route that does not involve one or more hazardous agents, causing risk to the health of production workers. These processes also require the use of organic solvents imposing additional environmental burden.

There remains a need in the industry for a safer, sustainable, and more economical system for the production of acetaminophen. The structural similarity of acetaminophen to p-aminophenol and p-aminobenzoic acid suggests that similar synthetic routes could lead to all three chemicals. P-aminophenol (PAP) is used in the production of pharmaceuticals, dyes, elastomers, photography, and prevention of ageing. Its polymer, polyp-aminophenol) is conductive and can be used to make sensors. P-aminobenzoic acid is used in dyes, crosslinking agents, polymers, nutritional supplements, and traditionally in sunscreen.

### SUMMARY OF THE INVENTION

The present disclosure provides compositions and methods for the biosynthetic production of medicaments such as acetaminophen. The present disclosure provides compositions and methods for the biosynthetic production of p-aminophenol. The present disclosure provides compositions and methods for the biosynthetic production of poly(p-aminophenol). The present disclosure provides compositions and methods for the biosynthetic production of p-aminobenzoic acid (PABA). The present disclosure provides methods to isolate and purify biologically derived acetaminophen.

Embodiments of the present invention comprise engineered organisms that produce acetaminophen. Embodiments of the present invention comprise engineered organisms that produce p-aminophenol. Embodiments of the present invention comprise engineered organisms that produce poly(p-aminophenol). Embodiments of the present invention comprise engineered organisms that produce PABA. The engineered organisms may include genetically tractable organisms such as plants, animals, bacteria, or fungi.

Embodiments of the present invention comprise methods of producing acetaminophen. The methods comprise providing a recombinant microorganism comprising an engineered acetaminophen biosynthesis pathway. The engineered microorganism may be used for the commercial production of acetaminophen via fermentation. Accordingly, in one embodiment the invention provides growing in suitable conditions, a recombinant microbial host cell comprising at least one DNA molecule encoding an enzyme(s) that catalyze a substrate to product conversion selected from the group consisting of:
i. chorismic acid to p-aminobenzoic acid (PABA) (pathway step a);
ii. p-aminobenzoic acid to p-aminophenol (pathway step b);
iii. p-aminophenol to acetaminophen (pathway step c),
   wherein the at least one DNA molecule is heterologous to said microbial host cell and wherein said microbial host cell produces acetaminophen. The method further includes cultivating the microorganism in a culture medium until a recoverable quantity of acetaminophen is produced and recovering the acetaminophen.

In another embodiment, a biotransformation method of producing acetaminophen is provided. The method comprises providing a recombinant microorganism comprising an engineered acetaminophen biosynthesis pathway. The engineered microorganism may be used for the commercial production of acetaminophen. Accordingly, in one embodiment the invention provides growing in suitable conditions, a recombinant microbial host cell comprising at least one DNA molecule encoding enzymes that catalyze both of the following substrate to product conversion:
i. p-aminobenzoic acid to p-aminophenol (pathway step b); and
ii. p-aminophenol to acetaminophen (pathway step c),
   wherein the at least one DNA molecule is heterologous to said microbial host cell, wherein PABA substrate is added to the growth culture, and wherein said microbial host cell produces acetaminophen. The method further includes cultivating the microorganism in a culture medium until a recoverable quantity of acetaminophen is produced and recovering the acetaminophen.

The present invention comprises methods of producing p-aminophenol. The methods comprise providing a recombinant microorganism comprising an engineered p-aminophenol biosynthesis pathway. The engineered microorganism may be used for the commercial production of p-aminophenol via fermentation. Accordingly, in one embodiment the invention provides growing in suitable conditions, a recombinant microbial host cell comprising at least one DNA molecule encoding an enzyme(s) that catalyze a substrate to product conversion selected from the group consisting of:
i. chorismic acid to p-aminobenzoic acid (PABA) (pathway step a); and
ii. p-aminobenzoic acid to p-aminophenol (pathway step b);
   wherein the at least one DNA molecule is heterologous to said microbial host cell and wherein said microbial host cell produces p-aminophenol. The method further includes cultivating the microorganism in a culture medium until a recoverable quantity of p-aminophenol is produced and recovering the p-aminophenol.

In another embodiment, a biotransformation method of producing p-aminophenol is provided. The method comprises providing a recombinant microorganism comprising an engineered p-aminophenol biosynthesis pathway. The engineered microorganism may be used for the commercial production of p-aminophenol. Accordingly, in one embodiment the invention provides growing in suitable conditions, a recombinant microbial host cell comprising at least one DNA molecule encoding enzymes that catalyze both of the following substrate to product conversion:
i. p-aminobenzoic acid to p-aminophenol (pathway step b);
   wherein the at least one DNA molecule is heterologous to said microbial host cell, wherein PABA substrate is added to the growth culture, and wherein said microbial host cell produces p-aminophenol. The method further includes cultivating the microorganism in a culture medium until a recoverable quantity of p-aminophenol is produced and recovering the p-aminophenol.

The present invention comprises methods of producing poly(p-aminophenol). The methods comprise providing a recombinant microorganism comprising an engineered p-aminophenol biosynthesis pathway. The engineered microorganism may be used for the commercial production of poly(p-aminophenol) via fermentation. Accordingly, in one embodiment the invention provides growing in suitable conditions, a recombinant microbial host cell comprising at least one DNA molecule encoding an enzyme(s) that catalyze a substrate to product conversion selected from the group consisting of:
i. chorismic acid to p-aminobenzoic acid (PABA) (pathway step a); and
ii. p-aminobenzoic acid to p-aminophenol (pathway step b);
   wherein the at least one DNA molecule is heterologous to said microbial host cell and wherein said microbial host cell produces poly(p-aminophenol). The method further includes cultivating the microorganism in a culture medium until a recoverable quantity of poly(p-aminophenol) is produced and recovering the poly(p-aminophenol). The polymer poly(p-aminophenol) is a brown pigment.

In another embodiment, a biotransformation method of producing poly(p-aminophenol) is provided. The method comprises providing a recombinant microorganism comprising an engineered p-aminophenol biosynthesis pathway. The engineered microorganism may be used for the commercial production of poly(p-aminophenol). Accordingly, in one embodiment the invention provides growing in suitable conditions, a recombinant microbial host cell comprising at least one DNA molecule encoding enzymes that catalyze both of the following substrate to product conversion:
i. p-aminobenzoic acid to p-aminophenol (pathway step b);
   wherein the at least one DNA molecule is heterologous to said microbial host cell, wherein PABA substrate is added to the growth culture, and wherein said microbial host cell produces poly(p-aminophenol). The method further includes cultivating the microorganism in a culture medium until a recoverable quantity of poly(p-aminophenol) is produced and recovering the poly(p-aminophenol).

The present invention comprises methods of producing p-aminobenzoic acid (PABA) via fermentation. The methods comprise providing a recombinant microorganism comprising an engineered p-aminobenzoic acid biosynthesis pathway. The engineered microorganism may be used for the commercial production of p-aminobenzoic acid via fermentation. In one embodiment, a method of producing p-aminobenzoic acid comprises providing a fermentation media comprising carbon substrate, contacting said media with a recombinant yeast microorganism expressing an engineered PABA biosynthetic pathway wherein said pathway comprises a chorismic acid to p-aminobenzoic acid (PABA) conversion; and culturing the yeast in conditions whereby PABA is produced. The method further includes cultivating the microorganism in a culture medium until a recoverable quantity of PABA is produced and recovering the PABA.

Some embodiments of the present invention comprise genetically engineered strains of yeast. In further embodiments, the yeast is S. cerevisiae. The present invention comprises engineered yeast strains that produce acetaminophen. Compositions of the present invention include yeast strains engineered with native and/or bacterial genes to produce acetaminophen from chorismic acid. S. cerevisiae is a preferred organism for biosynthetic production due to favorable consumer sentiment, the robust experience and infrastructure for scaling up fermentation, and lack of potential phage infection

The present invention comprises engineered yeast strains that produce p-aminophenol. Compositions of the present invention include yeast strains engineered with native and/or bacterial genes to produce p-aminophenol from chorismic acid via PABA.

The present invention comprises engineered yeast strains that produce poly(p-aminophenol). Compositions of the present invention include yeast strains engineered with native and/or bacterial genes to produce poly(p-aminophenol) from chorismic acid via PABA.

The present invention comprises engineered yeast strains that produce PABA. Compositions of the present invention include yeast strains engineered with native and/or bacterial genes to produce PABA from chorismic acid.

Strains of the present invention encode enzymes that convert the native yeast metabolite chorismic acid to p-aminobenzoic acid, p-aminobenzoic acid to p-aminophenol, and finally p-aminophenol to acetaminophen. In some embodiments, the engineered yeast strains encode enzymes that convert p-aminobenzoic acid to p-aminophenol, and p-aminophenol to acetaminophen. In some embodiments, the engineered yeast strains encode enzymes that convert chorismic acid to p-aminobenzoic acid and p-aminobenzoic acid to p-aminophenol, with p-aminophenol or poly(p-aminophenol) as the final product. In other embodiments, the engineered yeast strains encode enzymes that convert p-aminobenzoic acid to p-aminophenol, with p-aminophenol or poly(p-aminophenol) as the final product. In other embodiments, the strains encode enzymes that convert native yeast metabolite chorismic acid to p-aminobenzoic acid with p-aminobenzoic acid as the final product.

In one aspect, the engineered organisms have two to five genes or open reading frames under an inducible Gal promoter. The genes encode enzymes selected from the group consisting of aminodeoxychorismate lyase, aminodeoxychorismate synthase, glutamine amidotransferase, 4-aminobenzoate 1-monooxygenase, and N-hyroxyarylamine O-acetyltransferase. The genes may be native to the host, heterologous, or a combination. In certain embodiments, the two to five genes are selected from the group consisting of pabA, pabB, pabC, pabAB, pabBC, ABZ1, ABZ2, 4ABH, AAT and NhoA.

The enzymes that modify chorismic acid to form p-aminobenzoic acid (PABA) (pathway step a) are aminodeoxychorismate lyase and aminodeoxychorismate synthase. In some species such as E. coli, aminodeoxychorismate synthase is a heterodimeric complex composed of two proteins, glutamine amidotransferase (PabA) and 4-amino-4deoxychorismate synthase (PabB). In other species, such as Arabidopsis thaliana, the aminodeoxychorismate synthase is a monomeric enzyme. Therefore, the chorismic acid to p-aminobenzoic acid conversion may be encoded by three distinct genes such as pabA, pabB, and pabC or by genes that encode bifunctional proteins, such as those encoded by the genes pabAB, pabBC, or ABZ1.

The ABZ1 and ABZ2 genes from yeast encode a two protein complex. Though yeast natively encodes these two proteins, overexpression appears to be necessary for observable acetaminophen production. In some embodiments, PABA is exogenously added to the growth culture and the chorismic acid conversion step is bypassed. PABA is subsequently decarboxylated to form p-aminophenol (pathway step b). This step may be achieved by a 4-aminobenzoate 1-monoxygenase encoded by the 4ABH gene. The p-aminophenol intermediate may be the final product. Alternatively, the polymer form, poly(p-aminophenol) may be the final product. In growth medium, this production path results in the formation of a brown pigment which is poly(p-aminophenol).

In the final step of the acetaminophen synthesis, p-aminophenol is acetylated to produce acetaminophen (pathway step c) via the action of either N-hydroxyarylamine O-acetyltransferase encoded by NhoA or arylamine N-acetyltransferase encoded by AAT.

In another aspect, the engineered organisms have one to three open reading frames under an inducible Gal promoter that encode enzymes that convert chorismic acid to PABA. The open reading frames encode aminodeoxychorismate lyase and aminodeoxychorismate synthase. The enzymes may be encoded by heterologous genes. The heterologous genes may be pabA, pabB, pabC, pabAB, or pabBC. The genes may encode distinct mono-functional proteins or may encode bi-functional proteins.

The yeast strains described herein can be used to produce the popular medicament acetaminophen from chorismic acid via fermentation or from exogenously added PABA via biotransformation. The strains may be grown in a bioreactor and produce acetaminophen in the supernatant and cell pellet fraction. Subsequently, the acetaminophen can be purified. The strains encode enzymes that convert the native yeast metabolite chorismic acid to p-aminobenzoic acid, p-aminobenzoic acid to p-aminophenol, and p-aminophenol to acetaminophen. In some embodiments, the strains encode enzymes that convert exogenously added p-aminobenzoic acid to p-aminophenol, and p-aminophenol to acetaminophen.

The yeast strains described herein can be used to produce p-aminophenol, poly(p-aminophenol), and p-aminobenzoic acid from chorismic acid via fermentation. The yeast strains described herein can be used to produce p-aminophenol or poly(p-aminophenol) from exogenously added PABA via biotransformation. The strains may be grown in a bioreactor and produce p-aminophenol, poly(p-aminophenol), or p-aminobenzoic acid in the supernatant and cell pellet fraction. Subsequently, the p-aminophenol, poly(p-aminophenol), or p-aminobenzoic acid can be purified. The strains encode enzymes that convert the native yeast metabolite chorismic acid to p-aminobenzoic acid and p-aminobenzoic acid to p-aminophenol or poly(p-aminophenol). In some embodiments, the strains encode enzymes that convert exogenously added p-aminobenzoic acid to p-aminophenol or poly(p-aminophenol).

The present disclosure provides methods to isolate and purify acetaminophen. In one embodiment, the method is an evaporation process to concentrate and crystalize acetaminophen. In another embodiment, the method is an adsorption process utilizing specialized resins to isolate and recover acetaminophen. The present disclosure provides methods for the biosynthetic production of acetaminophen, p-aminophenol, poly(p-aminophenol) and p-aminobenzoic acid (PABA). Embodiments of the present invention comprise growing engineered yeast strains using more generalizable equipment based on fermentation technologies. As a result, the theoretical cost of the biological product could be as low as half the cost of the existing product, with additional benefits in reducing the capital costs of dedicated facilities, impact on the environment, safety of production workers, and potentially reduced impurities in the final products.

### BRIEF DESCRIPTION OF THE DRAWINGS

FIG. 1 shows the biosynthetic pathway encoded by strains of the present disclosure.

FIG. 2 shows the quantification of acetaminophen adsorbed versus the initial concentration of various resins.

### DETAILED DESCRIPTION OF THE INVENTION

Unless otherwise indicated, the practice of the disclosure involves conventional techniques commonly used in molecular biology, microbiology, protein purification, protein engineering, protein and DNA sequencing, and recombinant DNA fields, which are within the skill of the art. Such techniques are known to those of skill in the art, and are described in numerous standard texts and reference works. All patents, patent applications, articles and publications mentioned herein, both supra and infra, are hereby expressly incorporated herein by reference.

Unless defined otherwise herein, all technical and scientific terms used herein have the same meaning as commonly understood by one of ordinary skill in the art to which this disclosure belongs. Various scientific dictionaries that include the terms included herein are well known and available to those in the art. Although any methods and materials similar or equivalent to those described herein find use in the practice or testing of the disclosure, some preferred methods and materials are described. Accordingly, the terms defined immediately below are more fully described by reference to the specification as a whole. It is to be understood that this disclosure is not limited to the particular methodology, protocols, and reagents described, as these may vary, depending upon the context in which they are used by those of skill in the art.

As used herein, the singular terms “a”, “an,” and “the” include the plural reference unless the context clearly indicates otherwise.

Unless otherwise indicated, nucleic acids are written left to right in 5′ to 3′ orientation and amino acid sequences are written left to right in amino to carboxyl orientation, respectively.

Numeric ranges are inclusive of the numbers defining the range. It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The headings provided herein are not limitations of the various aspects or embodiments of the invention which can be had by reference to the specification as a whole.

The term “invention” or “present invention” as used herein is a non-limiting term and is not intended to refer to any single embodiment of the particular invention but encompasses all possible embodiments as described in the specification and the claims.

A modified microorganism for high efficient production of acetaminophen is provided herein. The present disclosure provides compositions and methods for an industrial fermentation process for the production of medicaments such as acetaminophen. The fermentation is conducted using various species, including yeast, bacteria, and fungi. The present disclosure also provides compositions and methods for an industrial biotransformation process for the production of medicaments such as acetaminophen. The microorganisms are genetically engineered to produce acetaminophen.

The term “microorganism” includes prokaryotic and eukaryotic microbial species from the Domains Archaea, Bacteria and Eucarya, the latter including yeast and filamentous fungi, protozoa, algae, or higher Protista. The terms “microbial cells” and “microbes” are used interchangeably with the term microorganism.

“Bacteria” or “eubacteria” refers to a domain of prokaryotic organisms. Bacteria include at least 11 distinct groups as follows: (1) Gram-positive (gram+) bacteria, of which there are two major subdivisions: (1) high G+C group (Actinomycetes, Mycobacteria, Micrococcus, others) (2) low G+C group (Bacillus, Clostridia, Lactobacillus, Staphylococci, Streptococci, Mycoplasmas); (2) Proteobacteria, e.g., Purple photosynthetic and non-photosynthetic Gram-negative bacteria (includes most “common” Gram-negative bacteria); (3) Cyanobacteria, e.g., oxygenic phototrophs; (4) Spirochetes and related species; (5) Planctomyces; (6) Bacteroides, Flavobacteria; (7) Chlamydia; (8) Green sulfur bacteria; (9) Green non-sulfur bacteria (also anaerobic phototrophs); (10) Radioresistant micrococci and relatives; (11) Thermotoga and Thermosipho thermophiles.

“Gram-negative bacteria” include cocci, non-enteric rods, and enteric rods. The genera of Gram-negative bacteria include, for example, Neisseria, Spirillum, Pasteurella, Brucella, Yersinia, Francisella, Haemophilus, Bordetella, Escherichia, Salmonella, Shigella, Klebsiella, Proteus, Vibrio, Pseudomonas, Bacteroides, Acetobacter, Aerobacter, Agrobacterium, Azotobacter, Spirilla, Serratia, Vibrio, Rhizobium, Chlamydia, Rickettsia, Treponema, and Fusobacterium.

“Gram positive bacteria” include cocci, nonsporulating rods, and sporulating rods. The genera of gram positive bacteria include, for example, Actinomyces, Bacillus, Clostridium, Corynebacterium, Erysipelothrix, Lactobacillus, Listeria, Mycobacterium, Myxococcus, Nocardia, Staphylococcus, Streptococcus, and Streptomyces.

Yeasts are eukaryotic microorganisms classified as members of the fungus kingdom and are estimated to constitute 1% of all described fungal species. Yeasts are unicellular, although some species may also develop multicellular characteristics by forming strings of connected budding cells known as pseudohyphae or false hyphae. Yeasts do not form a single taxonomic or phylogenetic grouping. The term “yeast” is often taken as a synonym for Saccharomyces cerevisiae, but the phylogenetic diversity of yeasts is shown by their placement in two separate phyla: the Ascomycota and the Basidiomycota.

The term “genus” is defined as a taxonomic group of related species according to the Taxonomic Outline of Bacteria and Archaea (Garrity, G. M., Lilburn, T. G., Cole, J. R., Harrison, S. H., Euzeby, J., and Tindall, B. J. (2007) The Taxonomic Outline of Bacteria and Archaea. TOBA Release 7.7, March 2007. Michigan State University Board of Trustees.

The term “species” is defined as a collection of closely related organisms with greater than 97% 16S ribosomal RNA sequence homology and greater than 70% genomic hybridization and sufficiently different from all other organisms so as to be recognized as a distinct unit.

As used herein, the term “isolated” when used in reference to a microbial organism is intended to mean an organism that is substantially free of at least one component as the referenced microbial organism is found in nature. The term includes a microbial organism that is removed from some or all components as it is found in its natural environment. The term also includes a microbial organism that is removed from some or all components as the microbial organism is found in non-naturally occurring environments. Therefore, an isolated microbial organism is partly or completely separated from other substances as it is found in nature or as it is grown, stored or subsisted in non-naturally occurring environments. Specific examples of isolated microbial organisms include partially pure microbes, substantially pure microbes and microbes cultured in a medium that is non-naturally occurring.

The term “gene” refers to a nucleic acid fragment that is capable of being expressed as a specific protein, optionally including regulatory sequences preceding (5′ non-coding sequences) and following (3′ non-coding sequences) the coding sequence. “Native gene” refers to a gene as found in nature with its own regulatory sequences. “Chimeric gene” refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature.

The term “endogenous gene” refers to a native gene in its natural location in the genome of an organism.

A “foreign gene” or “heterologous gene” refers to a gene not normally found in the host organism, but that is introduced into the host organism by gene transfer. Foreign genes can comprise native genes inserted into a non-native organism, or chimeric genes.

A “transgene” is a gene that has been introduced into the genome by a transformation procedure.

As used herein, the term “open reading frame” also referred to as “ORF” is the part of a reading frame that has the potential to code for a protein or peptide.

As used herein the term “coding sequence” refers to DNA sequence that code for a specific amino acid sequence. “Suitable regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, polyadenylation recognition sequences, RNA processing site, effector binding site and stem-loop structure. As used herein the term “codon degeneracy” refers to the nature in the genetic code permitting variation of the nucleotide sequence without affecting the amino acid sequence of an encoded polypeptide. The skilled artisan is well aware of the “codon-bias” exhibited by a specific host cell in usage of nucleotide codons to specify a given amino acid. Therefore, when synthesizing a gene for improved expression in a host cell, it is desirable to design the gene such that its frequency of codon usage approaches the frequency of preferred codon usage of the host cell.

The term “codon-optimized” as it refers to genes or coding regions of nucleic acid molecules for transformation of various hosts, refers to the alteration of codons in the gene or coding regions of the nucleic acid molecules to reflect the typical codon usage of the host organism without altering the polypeptide encoded by the DNA.

The term “promoter” refers to a DNA sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental or physiological conditions. Promoters which cause a gene to be expressed in most cell types at most times are commonly referred to as “constitutive promoters”. It is further recognized that since in most cases the exact boundaries of regulatory sequences have not been completely defined, DNA fragments of different lengths may have identical promoter activity.

As used herein, the term “genetically engineered” or “genetic engineering” or “genetic modification” involves the direct manipulation of an organism's genome using molecular and biotechnological tools and techniques. The present disclosure relates rational pathway design and assembly of biosynthetic genes, genes associated with operons, and control elements of such nucleic acid sequences, for the production of a desired metabolite, such as acetaminophen, in a microorganism.

As used herein, “metabolically engineered” can further include optimization of metabolic flux by regulation and optimization of transcription, translation, protein stability and protein functionality using genetic engineering and appropriate culture condition. The biosynthetic genes can be heterologous to the host (e.g., microorganism), either by virtue of being foreign to the host, or being modified by mutagenesis, recombination, or association with a heterologous expression control sequence in an endogenous host cell. Appropriate culture conditions are conditions such as culture medium pH, ionic strength, nutritive content, etc., temperature, oxygen, CO₂, nitrogen content, humidity, and other culture conditions that permit production of the compound by the host microorganism, i.e., by the metabolic action of the microorganism. Appropriate culture conditions are well known for microorganisms that can serve as host cells.

The term “recombinant microorganism” and “recombinant host cell” are used interchangeably herein and refer to microorganisms that have been genetically modified to express or over-express endogenous polynucleotides, or to express heterologous polynucleotides, such as those included in a vector, or which have an alteration in expression of an endogenous gene. By “alteration” it is meant that the expression of the gene, or level of a RNA molecule or equivalent RNA molecules encoding one or more polypeptides or polypeptide subunits, or activity of one or more polypeptides or polypeptide subunits is up regulated or down regulated, such that expression, level, or activity is greater than or less than that observed in the absence of the alteration. For example, the term “alter” can mean “inhibit,” but the use of the word “alter” is not limited to this definition.

The terms “metabolically engineered microorganism” and “modified microorganism” are used interchangeably herein and refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein. The introduction of genetic material into a host or parental microorganism of choice modifies or alters the cellular physiology and biochemistry of the microorganism. Through the introduction of genetic material the parental microorganism acquires new properties, e.g. the ability to produce a new, or greater quantities of, an intracellular metabolite.

As used herein, the term “non-naturally occurring” when used in reference to a microbial organism or microorganism of the invention is intended to mean that the microbial organism has at least one genetic alteration not normally found in a naturally occurring strain of the referenced species, including wild-type strains of the referenced species. Genetic alterations include, for example, modifications introducing expressible nucleic acids encoding metabolic polypeptides, other nucleic acid additions, nucleic acid deletions and/or other functional disruption of the microbial organism's genetic material. Such modifications include, for example, coding regions and functional fragments thereof, for heterologous, homologous or both heterologous and homologous polypeptides for the referenced species. Additional modifications include, for example, non-coding regulatory regions in which the modifications alter expression of a gene or operon. Exemplary metabolic polypeptides include enzymes or proteins within an acetaminophen biosynthetic pathway.

For example, the introduction of genetic material into a parental microorganism results in a new or modified ability to produce a chemical. The genetic material introduced into the parental microorganism contains gene, or parts of genes, coding for one or more of the enzymes involved in a biosynthetic pathway for the production of a chemical and may also include additional elements for the expression or regulation of expression of these genes, e.g. promoter sequences.

Those skilled in the art will understand that the genetic alterations, including metabolic modifications exemplified herein, are described with reference to a suitable host organism such as S. cerevisiae and their corresponding metabolic reactions or a suitable source organism for desired genetic material such as genes for a desired metabolic pathway. However, given the complete genome sequencing of a wide variety of organisms and the high level of skill in the area of genomics, those skilled in the art will readily be able to apply the teachings and guidance provided herein to essentially all other organisms. For example, the S. cerevisiae metabolic alterations exemplified herein can readily be applied to other species by incorporating the same or analogous encoding nucleic acid from species other than the referenced species. Such genetic alterations include, for example, genetic alterations of species homologs, in general, and in particular, orthologues, paralogs or non-orthologous gene displacements.

An orthologue is a gene or genes that are related by vertical descent and are responsible for substantially the same or identical functions in different organisms. For example, mouse epoxide hydrolase and human epoxide hydrolase can be considered orthologues for the biological function of hydrolysis of epoxides. Genes are related by vertical descent when, for example, they share sequence similarity of sufficient amount to indicate they are homologous, or related by evolution from a common ancestor. Genes can also be considered orthologues if they share three-dimensional structure but not necessarily sequence similarity, of a sufficient amount to indicate that they have evolved from a common ancestor to the extent that the primary sequence similarity is not identifiable. Genes that are orthologous can encode proteins with sequence similarity of about 25% to 100% amino acid sequence identity.

As used herein, the term “exogenous” or “heterologous” means that a biological function or material, including genetic material, of interest is not natural in a host strain. The term “native” means that such biological material or function naturally exists in the host strain or is found in a genome of a wild-type cell in the host strain.

Exogenous nucleic acid sequences involved in a pathway for production of acetaminophen can be introduced stably or transiently into a host cell using techniques well known in the art including, but not limited to, conjugation, electroporation, chemical transformation, transduction, transfection, and ultrasound transformation. For exogenous expression in E. coli or other prokaryotic cells, some nucleic acid sequences in the genes or cDNAs of eukaryotic nucleic acids can encode targeting signals such as an N-terminal mitochondrial or other targeting signal, which can be removed before transformation into prokaryotic host cells, if desired. For example, removal of a mitochondrial leader sequence led to increased expression in E. coli (Hoffmeister et al., J. Biol. Chem. 280:4329-4338 (2005)). For exogenous expression in yeast or other eukaryotic cells, genes can be expressed in the cytosol without the addition of leader sequence, or can be targeted to mitochondrion or other organelles, or targeted for secretion, by the addition of a suitable targeting sequence such as a mitochondrial targeting or secretion signal suitable for the host cells. Thus, it is understood that appropriate modifications to a nucleic acid sequence to remove or include a targeting sequence can be incorporated into an exogenous nucleic acid sequence to impart desirable properties. Furthermore, genes can be subjected to codon optimization with techniques well known in the art to achieve optimized expression of the proteins.

The term “expression” with respect to a gene sequence refers to transcription of the gene and, as appropriate, translation of the resulting mRNA transcript to a protein. Thus, as will be clear from the context, expression of a protein results from transcription and translation of the open reading frame sequence. The level of expression of a desired product in a host cell may be determined on the basis of either the amount of corresponding mRNA that is present in the cell, or the amount of the desired product encoded by the selected sequence. For example, mRNA transcribed from a selected sequence can be quantitated by PCR or by northern hybridization (see Sambrook et al., Molecular Cloning: A Laboratory Manual, Cold Spring Harbor Laboratory Press (1989)). Protein encoded by a selected sequence can be quantitated by various methods, e.g., by ELISA, by assaying for the biological activity of the protein, or by employing assays that are independent of such activity, such as western blotting or radioimmunoassay, using antibodies that are recognize and bind reacting the protein. See Sambrook et al., 1989, supra.

It is understood that the terms “recombinant microorganism” and “recombinant host cell” refer not only to the particular recombinant microorganism but to the progeny or potential progeny of such a microorganism. Because certain modifications may occur in succeeding generations due to either mutation or environmental influences, such progeny may not, in fact, be identical to the parent cell, but are still included within the scope of the term as used herein.

The term “wild-type microorganism” describes a cell that occurs in nature, i.e. a cell that has not been genetically modified. A wild-type microorganism can be genetically modified to express or overexpress a first target enzyme. This microorganism can act as a parental microorganism in the generation of a microorganism modified to express or overexpress a second target enzyme. In turn, the microorganism modified to express or overexpress a first and a second target enzyme can be modified to express or overexpress a third target enzyme.

Accordingly, a “parental microorganism” functions as a reference cell for successive genetic modification events. Each modification event can be accomplished by introducing a nucleic acid molecule in to the reference cell. The introduction facilitates the expression or overexpression of a target enzyme. It is understood that the term “facilitates” encompasses the activation of endogenous polynucleotides encoding a target enzyme through genetic modification of e.g., a promoter sequence in a parental microorganism. It is further understood that the term “facilitates” encompasses the introduction of heterologous polynucleotides encoding a target enzyme in to a parental microorganism.

As used herein the term “transformation” refers to the transfer of a nucleic acid fragment into a host organism, resulting in genetically stable inheritance. Host organisms containing the transformed nucleic acid fragments are referred to as “transgenic” or “recombinant” or “transformed” organisms.

The terms “plasmid”, “vector”, and “cassette” refer to an extra chromosomal element often carrying genes which are not part of the central metabolism of the cell, and usually in the form of circular double-stranded DNA fragments. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell. “Transformation cassette” refers to a specific vector containing a foreign gene and having elements in addition to the foreign gene that facilitates transformation of a particular host cell. “Expression cassette” refers to a specific vector containing a foreign gene and having elements in addition to the foreign gene that allow for enhanced expression of that gene in a foreign host.

The term “protein,” “peptide,” or “polypeptide” as used herein indicates an organic polymer composed of two or more amino acidic monomers and/or analogs thereof. As used herein, the term “amino acid” or “amino acidic monomer” refers to any natural and/or synthetic amino acids including glycine and both D or L optical isomers. The term “amino acid analog” refers to an amino acid in which one or more individual atoms have been replaced, either with a different atom, or with a different functional group. Accordingly, the term polypeptide includes amino acidic polymer of any length including full length proteins, and peptides as well as analogs and fragments thereof. A polypeptide of three or more amino acids is also called a protein oligomer or oligopeptide

The term “enzyme” as used herein refers to any substance that catalyzes or promotes one or more chemical or biochemical reactions, which usually includes enzymes totally or partially composed of a polypeptide, but can include enzymes composed of a different molecule including polynucleotides.

As used herein, an “enzymatically active domain” refers to any polypeptide, naturally occurring or synthetically produced, capable of mediating, facilitating, or otherwise regulating a chemical reaction, without, itself, being permanently modified, altered, or destroyed. Binding sites (or domains), in which a polypeptide does not catalyze a chemical reaction, but merely forms noncovalent bonds with another molecule, are not enzymatically active domains as defined herein. In addition, catalytically active domains, in which the protein possessing the catalytic domain is modified, altered, or destroyed, are not enzymatically active domains as defined herein. Enzymatically active domains, therefore, are distinguishable from other (non-enzymatic) catalytic domains known in the art (e.g., detectable tags, signal peptides, allosteric domains, etc.).

The term “homolog”, used with respect to an original enzyme or gene of a first family or species, refers to distinct enzymes or genes of a second family or species which are determined by functional, structural or genomic analyses to be an enzyme or gene of the second family or species which corresponds to the original enzyme or gene of the first family or species. Most often, homologs will have functional, structural or genomic similarities. Techniques are known by which homologs of an enzyme or gene can readily be cloned using genetic probes and PCR. Identity of cloned sequences as homolog can be confirmed using functional assays and/or by genomic mapping of the genes.

A protein has “homology” or is “homologous” to a second protein if the nucleic acid sequence that encodes the protein has a similar sequence to the nucleic acid sequence that encodes the second protein. Alternatively, a protein has homology to a second protein if the two proteins have “similar” amino acid sequences. Thus, the term “homologous proteins” is defined to mean that the two proteins have similar amino acid sequences.

The term “analog” or “analogous” refers to nucleic acid or protein sequences or protein structures that are related to one another in function only and are not from common descent or do not share a common ancestral sequence. Analogs may differ in sequence but may share a similar structure, due to convergent evolution. For example, two enzymes are analogs or analogous if the enzymes catalyze the same reaction of conversion of a substrate to a product, are unrelated in sequence, and irrespective of whether the two enzymes are related in structure.

An expression vector or vectors can be constructed to include one or more acetaminophen biosynthetic pathway encoding nucleic acids as exemplified herein operably linked to expression control sequences functional in the host organism. Expression vectors applicable for use in the microbial host organisms of the invention include, for example, plasmids, phage vectors, viral vectors, episomes and artificial chromosomes, including vectors and selection sequences or markers operable for stable integration into a host chromosome.

Additionally, the expression vectors can include one or more selectable marker genes and appropriate expression control sequences. Selectable marker genes also can be included that, for example, provide resistance to antibiotics or toxins, complement auxotrophic deficiencies, or supply critical nutrients not in the culture media. Expression control sequences can include constitutive and inducible promoters, transcription enhancers, transcription terminators, and the like which are well known in the art.

When two or more exogenous encoding nucleic acids are to be co-expressed, both nucleic acids can be inserted, for example, into a single expression vector or in separate expression vectors. For single vector expression, the encoding nucleic acids can be operationally linked to one common expression control sequence or linked to different expression control sequences, such as one inducible promoter and one constitutive promoter.

The transformation of exogenous nucleic acid sequences involved in a metabolic or synthetic pathway can be confirmed using methods well known in the art. Such methods include, for example, nucleic acid analysis such as Northern blots or polymerase chain reaction (PCR) amplification of mRNA, or immunoblotting for expression of gene products, or other suitable analytical methods to test the expression of an introduced nucleic acid sequence or its corresponding gene product. It is understood by those skilled in the art that the exogenous nucleic acid is expressed in a sufficient amount to produce the desired product, and it is further understood that expression levels can be optimized to obtain sufficient expression using methods well known in the art and as disclosed herein.

The term “fermentation” or “fermentation process” is defined as a process in which a microorganism is cultivated in a culture medium containing raw materials, such as feedstock and nutrients, wherein the microorganism converts raw materials, such as a feedstock, into products. Fermentation can be accomplished in batch or continuous production formats.

As used herein, the term “biotransformation” or “bioconversion” is the chemical modification made by an organism on a chemical compound.

As used interchangeably herein, the terms “activity” and “enzymatic activity” refer to any functional activity normally attributed to a selected polypeptide when produced under favorable conditions. Typically, the activity of a selected polypeptide encompasses the total enzymatic activity associated with the produced polypeptide. The polypeptide produced by a host cell and having enzymatic activity may be located in the intracellular space of the cell, cell-associated, secreted into the extracellular milieu, or a combination thereof.

As used herein, the term “acetaminophen biosynthesis” refers to a metabolic pathway that produces acetaminophen. The structure of acetaminophen is provided herein.

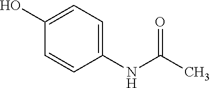

As used herein, the term “p-aminobenzoic acid biosynthesis” refers to a metabolic pathway that produces p-aminobenzoic acid, also referred to as PABA. The structure of p-aminobenzoic acid is provided herein.

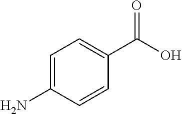

As used herein, the term “chorismic acid” is used interchangeably with the term for its anionic form “chorismate”.

The term “aminodeoxychorismate synthase” or “ADC synthase” refers to an enzyme that is part of a two protein complex which catalyzes the conversion of chorismic acid to p-aminobenzoic acid (PABA). It is a heterodimeric complex that catalyzes the chemical reaction chorismate+L-glutamine⇄4-amino-4-deoxychorismate+L-glutamate. These enzymes are available from a vast array of organisms. The enzyme may be, for example, encoded by the ABZ1 gene from, Saccharomyces cerevisiae. The enzyme may be, for example, encoded by pabA and pabB genes from Escherichia coli.

The term “aminodeoxychorismate lyase” refers to an enzyme that is part of a two protein complex which catalyzes the conversion of chorismic acid to p-aminobenzoic acid (PABA). Specifically, it catalyzes the chemical reaction 4-amino-4-deoxychorismate⇄4-aminobenzoate+pyruvate. This enzyme is available from a vast array of organisms. The enzyme may be, for example, encoded by the ABZ2 gene from Saccharomyces cerevisiae. The enzyme may be for example encoded by the pabC gene from Escherichia coli.

The term “4-aminobenzoate 1-monooxygenase” refers to an enzyme that catalyzes the decarboxylation of PABA to p-aminophenol. These enzymes are available from a vast array of organisms. The enzyme may be, for example, encoded by the 4ABH gene from Agaricus bisporus.

The term “N-hydroxyarylamine 0-acetyltransferase” refers to an enzyme that catalyzes the acetylation of p-aminophenol to produce acetaminophen. This enzyme is available from a vast array of organisms. The enzyme may be, for example, encoded by the NhoA gene from Escherichia coli.

The term “aryl N-acetyltransferase” refers to an enzyme that catalyzes the acetylation of p-aminophenol to produce acetaminophen. This enzyme is available from a vast array of organisms. The enzyme may be, for example, encoded by the AAT gene from Nocardia farcinica.

The first step (pathway step a) in acetaminophen biosynthesis is the modification of chorismic acid to p-aminobenzoic acid (PABA) which is catalyzed by a two-protein complex encoded by ABZ1 and ABZ2 in Saccharomyces cerevisiae. This step can by bypassed by the direct addition of PABA to the culture medium.

In the second step (pathway step b), PABA is decarboxylated to p-aminophenol by 4-aminobenzoate 1-monooxygenase. This may be encoded by the 4ABH gene from Agaricus bisporus.

The next step (pathway step c) in acetaminophen biosynthesis is the acetylation of p-aminophenol to acetaminophen. This step may be catalyzed by N-hydroxyarylamine O-acetyltransferase. The N-hydroxyarylamine O-acetyltransferase may be encoded by the NhoA gene from Escherichia coli. Alternatively, step c may be catalyzed by aryl N-acetyltransferase. Aryl N-acetyltransferase may be encoded by the AAT gene from Nocardia farcinica.

The term “volumetric productivity” or “production rate” is defined as the amount of product formed per volume of medium per unit of time. Volumetric productivity is reported in gram per liter per hour (g/L/h).

The term “yield” is defined as the amount of product obtained per unit weight of raw material and may be expressed as g product per g substrate (g/g). Yield may be expressed as a percentage of the theoretical yield. “Theoretical yield” is defined as the maximum amount of product that can be generated per a given amount of substrate as dictated by the stoichiometry of the metabolic pathway used to make the product.

The term “titer” is defined as the concentration of a substance in solution. Herein, it also refers to the concentration of product, usually expressed in g/L, upon completion of fermentation.

The term “filtration” is defined as any of various mechanical, physical, or biological operations that separate solids from fluids by adding a medium through which only the fluid can pass. The term “membrane filtration” refers to the use of a membrane to separate the solids from liquids.

The term “reverse osmosis” is defined as a process by which a solvent passes through a porous membrane in the direction opposite to that for natural osmosis when subjected to a hydrostatic pressure greater than the osmotic pressure. As used herein, reverse osmosis membranes can be used to concentrate liquid samples comprising acetaminophen, such that acetaminophen is retained in the retentate.

The term “resin” or “synthetic resin” refers to materials used to extract a molecule of interest from a complex mixture.

### Construction of Production Host

Recombinant organisms containing the necessary genes that will encode the enzymatic pathway for the biosynthetic production of acetaminophen may be constructed using techniques well known in the art. In the present invention, genes encoding the enzymes of one of the acetaminophen biosynthetic pathways of the invention, for example, ADC synthase, aminodeoxychorismate lyase, 4-aminobenzoate 1-monooxygenase, N-hydroxyarylamine O-acetyltransferase, or aryl N-acetyltransferase may be determined from the genomes of various organisms, as described above.

Methods of obtaining desired genes from a genome are common and well known in the art of molecular biology. For example, if the sequence of the gene is known, suitable synthetic genes are constructed by gene synthesis. Tools for codon optimization for expression in a heterologous host are readily available.

Once the relevant pathway genes are identified, the synthesized genes may be assembled into larger genetic constructs such as into suitable vectors. Means for this are well known in the art. Vectors or cassettes useful for the transformation of a variety of host cells are common and commercially available from gene synthesis companies such as DNA2.0, SGI-DNA, Invitrogen, and Genscript. Typically the vector or cassette contains sequences directing transcription and translation of the relevant gene, a selectable marker, and sequences allowing autonomous replication or chromosomal integration. Suitable vectors comprise a region 5′ of the gene which harbors transcriptional initiation controls and a region 3′ of the DNA fragment which controls transcriptional termination. Both control regions may be derived from genes homologous to the transformed host cell, although it is to be understood that such control regions may also be derived from genes that are not native to the specific species chosen as a production host.

### Engineered Microorganisms

According to one embodiment, a modified microorganism comprising a heterologous production system of acetaminophen is provided. The modified microorganisms may be yeast, bacteria, or fungi. The modified microorganisms may express heterologous proteins useful in the production of acetaminophen.

One embodiment of the present invention is a non-naturally occurring microorganism having an acetaminophen pathway and comprising at least four open reading frames encoding acetaminophen pathway enzymes expressed in a sufficient amount to produce acetaminophen, wherein said acetaminophen pathway comprises i. chorismic acid to p-aminobenzoic acid (PABA) (pathway step a); ii. p-aminobenzoic acid to p-aminophenol (pathway step b); and iii. p-aminophenol to acetaminophen (pathway step c).

Another embodiment of the present invention is a non-naturally occurring microorganism having an acetaminophen pathway and comprising at least two open reading frames encoding acetaminophen pathway enzymes expressed in a sufficient amount to produce acetaminophen, wherein said acetaminophen pathway comprises
i. p-aminobenzoic acid to p-aminophenol (pathway step b); and,
ii. p-aminophenol to acetaminophen (pathway step c);
   and wherein p-aminobenzoic acid is provided to the microorganism exogenously.

One embodiment of the present invention is a non-naturally occurring microorganism having an p-aminophenol pathway and comprising at least three open reading frames encoding p-aminophenol pathway enzymes expressed in a sufficient amount to produce p-aminophenol or poly(p-aminophenol), wherein said p-aminophenol pathway comprises
i. chorismic acid to p-aminobenzoic acid (PABA) (pathway step a); and
ii. p-aminobenzoic acid to p-aminophenol (pathway step b).

Another embodiment of the present invention is a non-naturally occurring microorganism having an p-aminophenol pathway and comprising at least one open reading frame encoding a p-aminophenol pathway enzyme expressed in a sufficient amount to produce p-aminophenol or poly(p-aminophenol), wherein said p-aminophenol pathway comprises p-aminobenzoic acid to p-aminophenol (pathway step b) and wherein p-aminobenzoic acid is provided to the microorganism exogenously.

One embodiment of the present invention is a non-naturally occurring microorganism having a p-aminobenzoic acid (PABA) pathway and comprising at least two open reading frames encoding p-aminobenzoic acid (PABA) pathway enzymes expressed in a sufficient amount to produce PABA, wherein said PABA pathway comprises chorismic acid to p-aminobenzoic acid (PABA) (pathway step a).

In some embodiments of the present invention, the enzyme that converts chorismic acid to p-aminobenzoic acid (PABA) is a two protein complex comprising aminodeoxychorismate lyase and bifunctional PabA-PabB ADC synthase. The two protein complex may be encoded by native host genes. Alternatively, the two protein complex may be overexpressed in the host. In other embodiments of the present invention, the enzyme that converts p-aminobenzoic acid to p-aminophenol is 4-aminobenzoate 1-monooxygenase. In other embodiments of the present invention, the enzyme that converts p-aminophenol to acetaminophen is N-hydroxyarylamine O-acetyltransferase. In yet other embodiments, the enzyme that converts p-aminophenol to acetaminophen is arylamine N-acetyltransferase.

Examples of exogenous genes that may be expressed in modified microorganisms of the present invention include genes that encode enzymes such as aminodeoxychorismate lyase, bifunctional PabA-PabB ADC synthase, 4-aminobenzoate 1-monooxygenase, N-hydroxyarylamine O-acetyltransferase, and arylamine N-acetyltransferase. These genes may be derived from animals, plants, bacteria, yeast, or fungi. Further, said nucleic acid encoding molecules (e.g., genes) may be codon optimized for use in an organism of interest.

In some embodiments, the modified microorganism is a yeast cell. In some embodiments, the recombinant microorganisms may be yeast recombinant microorganisms of the Saccharomyces clade. In certain embodiments, the modified yeast may be Saccharomyces cerevisiae. The S. cerevisiae may be strain S288C or a derivative thereof.

The modified yeast may encode native ABZ1 and ABZ2 genes that encode ADC synthase (bifunctional PabA-PabB) and aminodeoxychorismate lyase (PabC), respectively. Alternatively, these genes may be overexpressed. The ABZ1 gene may encode a polypeptide comprising SEQ ID NO: 1 or the active domain thereof. The ABZ2 gene may encode a polypeptide comprising SEQ ID NO: 2 or the active domain thereof.

The modified yeast may encode distinct PabA, PabB, and PabC enzymes in three distinct open reading frames. Alternatively, the modified yeast may encode two distinct proteins PabAB and PabC or PabA and PabBC from two distinct open reading frames. In another embodiment, the modified yeast may encode PabABC from one open reading frame. The genes may be derived from bacteria. Examples include E. coli and Agaricus bisporus.

The modified yeast may encode at least one heterologous gene selected from the group consisting of 4-aminobenzoate 1-monooxygenase, N-hydroxyarylamine O-acetyltransferase, and arylamine N-acetyltransferase. The heterologous genes may be derived from bacteria, yeast, fungi, plants, or animals. The 4-aminobenzoate 1-monooxygenase may be an Agaricus bisporus 4ABH gene and encode a polypeptide comprising SEQ ID NO: 3 or the active domain thereof. The N-hydroxyarylamine O-acetyltransferase may be an Escherichia coli NhoA gene and encode a polypeptide comprising SEQ ID NO: 4 or the active domain thereof. The arylamine N-acetyltransferase may be a Nocardia Farcinica AAT gene and encode a polypeptide comprising SEQ ID NO: 5 or the active domain thereof.

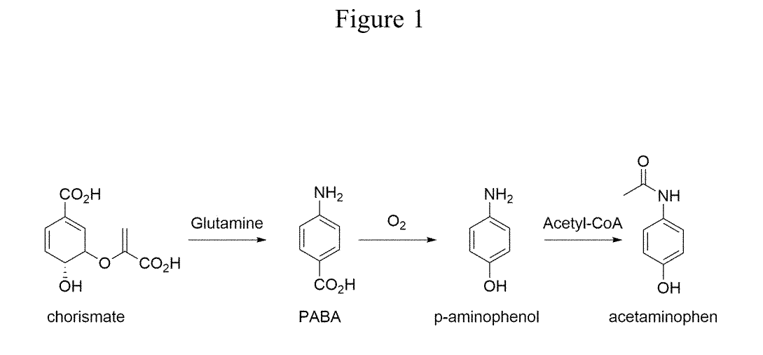

The biosynthetic pathway encoded by these strains is described in FIG. 1. The native metabolite, chorismic acid, is modified to form p-aminobenzoic acid (PABA) by a two-protein complex (encoded by ABZ1 and ABZ2). Strains pal and pa3 differ only by the presence and absence (respectively) of these two genes and only pal produces the final product without exogenous addition of PABA. PABA is then decarboxylated by 4-aminobenzoate 1-monooxygenase to form the unstable intermediate p-aminophenol. P-aminophenol is acetylated by either N-hydroxyarylamine O-acetyltransferase (NhoA from E. coli; pa1) or arylamine N-acetyltransferase (AAT from N. farcinica; pa3) to yield acetaminophen.

### Methods of Production

The present disclosure provides methods for the biosynthetic production of acetaminophen using engineered microorganisms of the present invention.

In one embodiment, a method of producing acetaminophen is provided. The method comprises providing a fermentation media comprising carbon substrate; contacting said media with a recombinant yeast microorganism expressing an engineered acetaminophen biosynthetic pathway wherein said pathway comprises the following substrate to product conversions: i. chorismic acid to p-aminobenzoic acid (PABA) (pathway step a); ii. p-aminobenzoic acid to p-aminophenol (pathway step b); iii. p-aminophenol to acetaminophen (pathway step c); and culturing the yeast in conditions whereby acetaminophen is produced. In methods of the present inventions, the substrate to product conversion of pathway step “a” is performed by aminodeoxychorismate lyase and ADC synthase; the substrate to product conversion of pathway step “b” is performed by a 4-aminobenzoate 1-monoygenase enzyme; and the substrate to product conversion of pathway step “c” is performed by an enzyme selected from the group consisting of N-hydroxyarylamine O-acetyltransferase and arylamine N-acetyltransferase. The method further includes cultivating the microorganism in a culture medium until a recoverable quantity of acetaminophen is produced and recovering the acetaminophen.

In another embodiment, a method of producing acetaminophen via biotransformation is provided. The method comprises providing a media comprising carbon substrate and exogenously added PABA; contacting said media with a recombinant yeast microorganism expressing an engineered acetaminophen biosynthetic pathway wherein said pathway comprises the following substrate to product conversions: i. p-aminobenzoic acid to p-aminophenol (pathway step b); ii. p-aminophenol to acetaminophen (pathway step c); and culturing the yeast in conditions whereby acetaminophen is produced. In methods of the present inventions, the substrate to product conversion of pathway step “b” is performed by a 4-aminobenzoate 1-monoygenase enzyme; and the substrate to product conversion of pathway step “c” is performed by an enzyme selected from the group consisting of N-hydroxyarylamine O-acetyltransferase and arylamine N-acetyltransferase. The method further includes cultivating the microorganism in a culture medium until a recoverable quantity of acetaminophen is produced and recovering the acetaminophen.

In one embodiment, a method of producing p-aminophenol or poly(p-aminophenol) is provided. The method comprises providing a fermentation media comprising carbon substrate; contacting said media with a recombinant yeast microorganism expressing an engineered p-aminophenol biosynthetic pathway wherein said pathway comprises the following substrate to product conversions: i. chorismic acid to p-aminobenzoic acid (PABA) (pathway step a); and ii. p-aminobenzoic acid to p-aminophenol (pathway step b); and culturing the yeast in conditions whereby p-aminophenol or poly(p-aminophenol) is produced. In methods of the present inventions, the substrate to product conversion of pathway step “a” is performed by aminodeoxychorismate lyase and ADC synthase and the substrate to product conversion of pathway step “b” is performed by a 4-aminobenzoate 1-monoygenase enzyme. The method further includes cultivating the microorganism in a culture medium until a recoverable quantity of p-aminophenol or poly(p-aminophenol) is produced and recovering the p-aminophenol or poly(p-aminophenol).

In another embodiment, a method of producing p-aminophenol or poly(p-aminophenol) via biotransformation is provided. The method comprises providing a media comprising carbon substrate and exogenously added PABA; contacting said media with a recombinant yeast microorganism expressing an engineered p-aminophenol biosynthetic pathway wherein said pathway comprises a p-aminobenzoic acid to p-aminophenol conversion; and culturing the yeast in conditions whereby p-aminophenol or poly(p-aminophenol) is produced. In methods of the present inventions, the p-aminobenzoic acid to p-aminophenol conversion is performed by a 4-aminobenzoate 1-monoygenase enzyme. The method further includes cultivating the microorganism in a culture medium until a recoverable quantity of p-aminophenol or poly(p-aminophenol) is produced and recovering the p-aminophenol or poly(p-aminophenol).

In one embodiment, a method of producing p-aminobenzoic acid (PABA) is provided. The method comprises providing a fermentation media comprising carbon substrate; contacting said media with a recombinant yeast microorganism expressing an engineered PABA biosynthetic pathway wherein said pathway comprises a chorismic acid to p-aminobenzoic acid (PABA) conversion (pathway step a); and culturing the yeast in conditions whereby PABA is produced. In methods of the present inventions, the substrate to product conversion of chorismic acid to p-aminobenzoic acid is performed by aminodeoxychorismate lyase and ADC synthase. The method further includes cultivating the microorganism in a culture medium until a recoverable quantity of PABA is produced and recovering the PABA.

Some embodiments of the present invention comprise yeast strains (designated pa1, pa2, and pa3) derived from S. cerevisiae strain S288C. Each encodes at least 2 foreign genes under inducible Gal promoters. The specific proteins encoded by each strain and their sequences, source, and accession numbers are provided in Table 1. The genes for these proteins are synthesized with yeast-optimized codon usage, assembled into singular genetic cassettes, and then inserted into the HO locus of S288C under URA2 selection.

When grown in SC Minimal Broth with 2% raffinose and 1% galactose, strains pa1 and pa3 produce around 1 mM acetaminophen in both supernatants and in cell pellets, as indicated by LC-MS analysis. When supplemented with PABA, all three strains produce the desired product with the highest yield from strain pa3. When grown in high concentrations of PABA, strain pa3 produces at least 10 mM acetaminophen.

Chorismate and the cofactors involved in the acetaminophen pathway are universal to all organisms, and thus the host organism could be any genetically tractable organism (plants, animals, bacteria, or fungi). Among yeast, other species such as S. pombe or P. pastoris are plausible alternatives. Within the S. cerevisiae species, other strains more amenable to large-scale productions, such as CENPalpha, may be utilized.

The Gal promoter used in embodiments of the present invention could be replaced with constitutive promoters, or other chemically-inducible, growth phase-dependent, or stress-induced promoters. Heterologous genes of the present invention may be genomically encoded or alternatively encoded on plasmids or yeast artificial chromosomes (YACs). All genes introduced could be encoded with alternate codon usage without altering the biochemical composition of the system. All enzymes used in embodiments of the present invention have extensive orthologues in the biosphere that could be encoded as alternatives.

The ABZ1 and A13Z2 genes could be replaced with orthologues from other yeast. Many such orthologues exist. Similarly, there are three-gene routes from chorismate to PABA, many from bacteria including E. coli which could be used. In addition, NhoA from E. coli and the Nocardia acetyltransferase have many orthologues which could be used.

### Culture Conditions

The growth medium used for production of acetaminophen by the engineered strains may be any media known in the art. Specifically, in particular embodiments the growth media may be Teknova SC Minimal Broth with Raffinose supplemented with 1% galactose.

### Purification Protocol

Once the various strains of the present invention are cultured in a bioreactor, biologically derived acetaminophen produced remains in solution in the fermentation broth along with other constituents from the fermentation process. The acetaminophen needs to be isolated and purified prior to use in any product formulation. The present disclosure provides methods to isolate and purify acetaminophen.

Embodiments of the present invention comprise methods for the isolation and purification of biologically derived acetaminophen produced from engineered microbial organisms cultured in a bioreactor. Methods for the isolation and purification may comprise solid phase extraction, evaporation, or adsorption. Methods of the present invention comprise producing a cell-free broth. This may be accomplished by methods known in the art, such as but not limited to centrifugation or filtration. In some embodiments, the isolation and purification process is an evaporation process to concentrate and crystallize acetaminophen from culture broth. In another embodiment, the process is an adsorption process using specialized resins to isolate and recover acetaminophen. In yet another embodiment, the purification process comprises solid phase extraction of acetaminophen. The solid phase may be any known in the art, for example, silica particles. The surface of the silica particles, also referred to as diatomaceous earth, may be coated by drying after treatment with polystyrene dissolved in tetrahydrofuran or similar solvent.

The present disclosure provides a concentration/evaporation process based on the solubility of acetaminophen. Acetaminophen is known to have a low solubility at room temperature that increases with temperature. Methods of the present invention comprise using a combination of membrane filtration and evaporation, to increase the concentration of acetaminophen in the fermentation broth by reducing the volume. Reduction of fermentation broth volume is achieved by removing water via evaporation and/or filtration, then cooling the liquid to cause the acetaminophen to crystalize. The resulting crystal slurry is filtered and the acetaminophen crystals recovered. The membranes used in the process can be varied, assuming that a compatible membrane with the same permeability qualities is employed.

The evaporation process methods of the present invention comprise (a) centrifuging a fermentation broth that comprises biologically derived acetaminophen to produce a cell pellet (b) decanting and retaining the supernatant (c) heating the supernatant to 80° C. to evaporate liquid and concentrate the supernatant (d) cooling the remaining solution (e) filtering the solution (f) collecting the acetaminophen crystals and (g) drying the crystals. In some embodiments, a wash step may be performed by repeating process steps a-g after re-solubilizing the acetaminophen crystals in distilled water. An optional step in the evaporation process is including a membrane filtration step to reduce the evaporation time and reduce the amount of heat required in the system. In some embodiments, the membrane is reverse osmosis membrane. In other embodiments, the membrane is a nano-filtration membrane.

The present disclosure provides adsorption methods using specialized resins to bind acetaminophen from fermentation broth. Resins may be chosen for their ability to remove aromatic compounds, such as phenol. The resins used can be replaced with other hydrophobic resins. Overall, six resins were tested (See Example 3) and two (XAD4 and SP825L) yielded the best results. Other compatible resins may be used. The adsorption process described herein is a batch process in which the resin is mixed directly into the supernatant. Alternatively, a resin bed column can be used instead with similar or better results, depending on the size of the resin bed.

Adsorption process methods of the present invention comprise (a) centrifuging a fermentation broth that comprises biologically derived acetaminophen to produce a cell pellet (b) decanting and retaining the supernatant (c) adding adsorbent resin to the supernatant (d) mixing the solution (e) equilibrating the solution (f) decanting the solution while retaining the resin (g) washing the resin with methanol to elute acetaminophen (h) decanting the methanol using filter paper (i) drying the methanol-acetaminophen solution to form acetaminophen crystals and (k) collecting the acetaminophen crystals. In some embodiments, a wash step may be performed by repeating process steps a-k after re-solubilizing the acetaminophen crystals in distilled water.

### EXAMPLES

### Example 1

### Strain Development

Three yeast prototypes constructed and successfully tested (strains pa1, pa2, and pa3) are derived from S. cerevisiae strain S288C (Table 1). Each encodes 2 or 4 genes under inducible Gal promoters. The specific proteins encoded by each strain and their sequences, source, and accession numbers are provided in Table 1. The genes for these proteins were synthesized with yeast optimized codon usage, assembled into singular genetic cassettes, and then inserted into the HO locus of S288C under URA2 selection.

TABLE 1
Strain constructs

| Strain | Accession No. | Source | Name | Enzyme |
|---|---|---|---|---|
| pa1 | NP_415980 | Escherichia coli | EcNhoA | N-hydroxyarylamine |
| | | | | O-acetyltransferase |
| | BAA07468 | Agaricus bisporus | Ab4ABH | 4-aminobenzoate 1- |
| | | | | monooxygenase |
| | CAA96313 | Saccharomyces cerevisiae | ABZ1 | bifunctional PabA- |
| | | | | PabB ADC synthase |
| | DAA10190 | Saccharomyces cerevisiae | ABZ2 | aminodeoxychorismate |
| | | | | lyase |
| pa2 | 3D9W_A | Nocardia Farcinica | NfAAT | Arylamine N- |
| | | | | Acetyltransferase |
| | BAA07468 | Agaricus bisporus | Ab4ABH | 4-aminobenzoate 1- |
| | | | | monooxygenase |
| | CAA96313 | Saccharomyces cerevisiae | ABZ1 | bifunctional PabA- |
| | | | | PabB ADC synthase |
| | DAA10190 | Saccharomyces cerevisiae | ABZ2 | aminodeoxychorismate |
| | | | | lyase |
| pa3 | NP_415980 | Escherichia coli | EcNhoA | N-hydroxyarylamine |
| | | | | O-acetyltransferase |
| | BAA07468 | Agaricus bisporus | Ab4ABH | 4-aminobenzoate 1- |
| | | | | monooxygenase |

The biosynthetic pathway encoded by these strains is described in FIG. 1. The ABZ1 and ABZ2 genes encode a two protein complex that modifies chorismic acid to form p-aminobenzoic acid (PABA). Though yeast natively encodes these two proteins, overexpression appears to be necessary for observable acetaminophen production. This step may be bypassed by exogenous addition of PABA, as with strain pa3. PABA is subsequently decarboxylated by to form p-aminophenol. This step may be achieved by a 4-aminobenzoate 1-monoxygenase encoded by the 4ABH gene. The p-aminophenol intermediate is unstable within the cell and in growth medium resulting in the formation of a brown pigment. In the final step of the synthesis, p-aminophenol is acetylated to produce acetaminophen via the action of either N-hydroxyarylamine O-acetyltransferase encoded by NhoA or arylamine N-acetyltransferase encoded by AAT

Strains pa1 and pa3 differ only in the presence and absence (respectively) of the ABZ1 and ABZ2 genes. Only pal produces the product without exogenous feeding of PABA when compared to pa3. PABA is subsequently decarboxylated to form p-aminophenol. This step is performed by the 4ABH gene from Agaricus bisporus. The p-aminophenol intermediate is unstable within the cell and in growth medium resulting in the formation of a brown pigment. Therefore, feeding or quantification of this intermediate has not yet been explored. In the last step, p-aminophenol is acetylated to produce acetaminophen via the action of either NhoA from E. coil (pa1) or arylamine N-Acetyltransferase from Nocardia farcinica (pa3). When grown in SC Minimal Broth with 2% Raffinose and 1% galactose, strains pa1 and pa3 produce around 1 mM acetaminophen in both supernatants and cells pellets, as indicated by LC-MS analysis. When supplemented with PABA, all three strains produce the desired product with the highest yield from strain pa3. When grown with high concentrations of PABA, strain pa3 produces at least 10 mM acetaminophen.

### Example 2

### Production

To test strains for chemical production, cells were grown in medium and then prepared for analysis by LC-MS. Medium containing 2% raffinose minus uracil from Teknova was prepared according to the manufacturer's protocol and is referred to as “Pregrowth Medium”. The same medium supplemented with 1% galactose was prepared as “Induction Medium”. Plastic 24-well plates were filled with 3 mL of Pregrowth Medium and then inoculated with frozen yeast stocks. The blocks were grown with shaking at 30° C. for 48 hours to generate saturated pregrowth cultures. These cultures were diluted 10 L into 4 mL of Induction Medium in additional 24-well plates to induce expression of the expressed genes. In some experiments, beta-alanine, histidine, or aspartate were also included in the induction culture. The plates were grown with shaking at 30° C. for 48 hours to generate saturated induction cultures. The plates were then subjected to centrifugation at 6000 rcf for 5 min to pellet the cells. Aliquots of clarified supernatant were transferred to a 96-well plate for analysis by LC-MS. The cells were then centrifuged a second time and the remainder of the supernatant removed. To prepare pellet extracts, 1 mL of room temperature methanol was added to each well and the cells were resuspended by shaking for 5 min. The plate was again centrifuged to remove cell debris, and the clarified extract was transferred to a 96-well plate. The collected samples were analyzed in 2 microliter aliquots by LC-MS on a Waters Xevo-G2-XS-QTof with a C18 column and a mobile phase gradient between 0.1% formic acid and acetonitrile with 0.1% formic acid. Two technical replicates of the induction, extraction, and analysis steps were performed for each experimental condition.

### Example 3

### Evaporation process for Acetaminophen Purification

Methods for capturing and purifying biologically-derived acetaminophen are described here. Because there is no expected difference between chemically-derived and biologically-derived acetaminophen, testing was done with spent fermentation broth spiked with chemically derived acetaminophen to increase its concentration to what will likely be seen in bioreactors.

One process tested was a concentration/evaporation process based on the solubility of acetaminophen. Acetaminophen is known to have a low solubility at room temperature that increases with temperature. By using a combination of membrane filtration and evaporation, the concentration of acetaminophen in the fermentation broth was increased by reducing the volume by removing water by evaporation and/or filtration, then cooling the liquid to cause the acetaminophen to crystalize. The resulting crystal slurry was then filtered and the acetaminophen crystals were recovered.

The evaporation process is as follows:

1. Centrifuge the fermentation broth to pellet the cells. Decant and retain the supernatant.

2. Concentrate the supernatant by evaporation by heating to 80° C. Continue concentration until reaching 10% of original volume. This target is not critical, but a greater concentration will result in a higher yield.

3. Chill the remaining solution in an ice bath. At this point, acetaminophen crystals should start forming. If not, scratch the container surface gently with glass rod to initiate crystal formation.

4. Filter the solution using a Buchner funnel and collect the acetaminophen crystals.

5. Dry the crystals.

6. If necessary, a wash may be performed, repeating process steps (1-5) after re-solubilizing the powder in a minimal amount of distilled water.

An optional step to the above process is to include a membrane filtration step after centrifugation of the culture in order to reduce the evaporation time and reduce the amount of heat required in the system. Two different membrane techniques were evaluated: (A) A reverse osmosis membrane, DOW FILMTEC XLE, can be used to concentrate the broth while retaining acetaminophen in the retentate. This allowed a 4× reduction in volume by filtration. The retentate was then evaporated as described above. The pre-filtration reduced the time required for evaporation. The resultant crystals from this process had a lighter brown color, mainly due to the reduced time of heating. (B) A nano-filtration membrane, Tri-Sep TS40, was also evaluated to concentrate the broth. This membrane allowed acetaminophen quantitatively into the permeate and rejected the color-causing compounds. The nano-filtration reduced the volume by 30%. The permeate was then evaporated as described above. This approach has the benefit of removing color from the resulting crystals.

The results of these processes depend upon the initial concentration of acetaminophen and the percent volume reduction. Tested at 2-2.5 g/L initial acetaminophen (APAP) concentration, process (1) yielded a 110% recovery of dry powder of a brownish tint. Adding in optional step (A), reduced the yield to 66%, but resulted in a lighter colored powder. Optional step (B) gave a white powder with a yield of 56%. The recovery for process (1) is greater than 100% due to due to UV-absorbing residual components and impurities from the broth being counted as APAP.

TABLE 2
Results for Process (1)

| | | | Vol- | Vol- | g | % |
|---|---|---|---|---|---|---|
| | | | ume | ume | Dry | recov- |
| | Starting | | after | after | Pow- | ery |
| | Vol- | g | Filtra- | Evap- | der | of |
| | ume | APAP | tion | oration | recov- | added |
| Process | (mL) | added | (mL) | (mL) | ered | APAP |
| Evaporation | 200 | 0.5 | — | 15 | 0.55 | 110 |
| (A) Membrane + | 200 | 0.5 | 125 | 20 | 0.33 | 66 |
| Evaporation | | | | | | |
| (B) TS40 + | 100 | 0.25 | 70 | 10 | 0.14 | 56 |
| evaporation | | | | | | |
| Membrane | | | | | | |

### Example 4

### Resin Adsorption Process for Acetaminophen Purification

An alternative process tested for acetaminophen production is an adsorption process using specialized resins to bind the acetaminophen from the broth. Resins were chosen for their ability to remove aromatic compounds, such as phenol. Six resins were tested (Table 3) All resins were hydrophobic styrene-divinylbenzene based.

The test procedure was as follows:
1. Centrifuge the fermentation broth to pellet the cells. Decant and retain the supernatant.
2. Add adsorbent resin to the supernatant and mix thoroughly. Add enough resin to achieve the target APAP recovery.
3. Allow the solution to come to equilibrium. This will take 2-3 hours. Carefully decant the solution while retaining the resin. Filter paper can be used to aid in retaining the resin.
4. Wash the resin with methanol to elute the acetaminophen. A smaller amount of methanol will be required than the original solution volume due to the greater solubility of acetaminophen in methanol. Allow the methanol and resin to equilibrate.
5. Carefully decant the methanol using filter paper. The filtered resin may be washed with more methanol to increase the recovery. The resin may then be used for another adsorption cycle.
6. Allow the methanol-APAP solution to evaporate to dryness. Crystals should appear as the solution dries.
7. Collect the acetaminophen crystals.
8. If necessary, a water wash may be performed to further purify the crystals. Follow step (6) of Process (1).

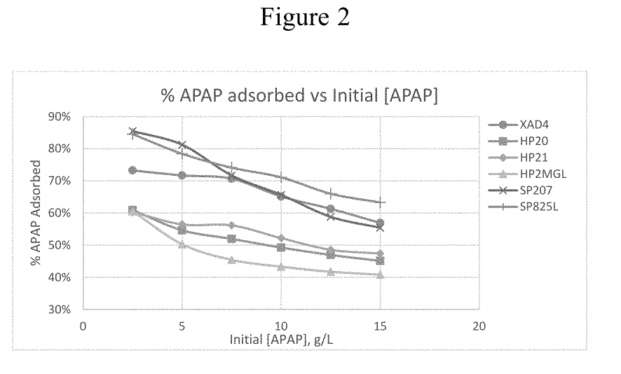

Initial testing was performed with water-based solutions of acetaminophen. Acetaminophen concentrations were quantified using a UV spectrophotometer assay, reading absorbance at 250 nm. XAD4 and SP825L yielded the best results (Table 3, FIG. 2) based upon their Freundlich constants. Again, the recovery will vary based on the initial concentration of acetaminophen and the amount of resin used. Using a 10% w/v resin/solution, 57% of the acetaminophen was adsorbed by XAD4 at an initial APAP concentration of 15 g/L, and 77% at 2.5 g/L. SP825L preformed slightly better, with yields of 63% and 81%, respectively. This process yielded white acetaminophen crystals.

TABLE 3
Freundlich Constants for Resins
Freundlich Constants for Resins

| | K | 1/n | n | R² |
|---|---|---|---|---|
| XAD4 | 0.0269 | 0.6756 | 1.4802 | 0.9700 |
| HP20 | 0.0155 | 0.7047 | 1.4190 | 0.9995 |
| HP21 | 0.0158 | 0.7456 | 1.3412 | 0.9914 |
| HP2MGL | 0.0147 | 0.6329 | 1.5800 | 0.9940 |
| SP207 | 0.0371 | 0.4455 | 2.2447 | 0.9764 |
| SP825L | 0.0372 | 0.5680 | 1.7606 | 0.9968 |

Samples of XAD4 and SP825L resins that had been loaded with APAP were washed with methanol. 93-94% of the acetaminophen was eluted from the resin. Samples of methanol were evaporated and resulted in an 86% recovery of acetaminophen from XAD4 and 75% recovery from SP825L. (Table 4).

TABLE 4
Recovery of Acetaminophen by Methanol Extraction

| | | g APAP | % APAP | | % APAP |
|---|---|---|---|---|---|
| | | extracted | extracted | | recovery |
| | g APAP in | in | extracted | g APAP | from |
| Resin | resin | Methanol | from resin | recovered | Methanol |
| XAD4 | 0.1707892 | 0.15898 | 93.09 | 0.123 | 86.0 |
| SP825L | 0.1898836 | 0.178256 | 93.88 | 0.1203 | 75.0 |

Testing was also performed with acetaminophen in spent fermentation broth using the same two resins. The maximum concentration of acetaminophen in the spent broth was 0.821 g/L assuming the UV absorbance of the broth correlated with APAP concentration. More acetaminophen was added to reach expected targets for testing. SP825L was still the better resin here, with 63% of the added acetaminophen adsorbed at 15 g/L, and 97% from the spent broth. XAD4 adsorbed 58% and 88%, respectively, at the same concentrations (Tables 5 and 6).

TABLE 5
Acetaminophen Adsorption by XAD4 in Spent Broth
XAD4 (adjusted)

| | | Initial | Final | | | |
|---|---|---|---|---|---|---|
| Mass | | APAP | APAPA | | | |
| Adsorb- | Vol- | Concen- | Concen- | grams | q (g APAP | % |
| ent | ume | tration | tration | APAP | adsorbed/g | APAP |
| (g) | (mL) | (g/L) | (g/L) | Adsorbed | adsorbent) | Adsorbed |
| 2 | 20 | 0.821 | 0.096 | 0.0145 | 0.0073 | 88.33 |
| 2 | 20 | 3.321 | 0.774 | 0.0509 | 0.0255 | 76.71 |
| 2 | 20 | 5.821 | 1.783 | 0.0808 | 0.0404 | 69.36 |
| 2 | 20 | 10.821 | 4.340 | 0.1296 | 0.0648 | 59.89 |
| 2 | 20 | 15.821 | 6.670 | 0.1830 | 0.0915 | 57.84 |

TABLE 6
Acetaminophen Adsorption by SP825L in Spent Broth
SP825L (adjusted)

| | | Initial | Final | | | |
|---|---|---|---|---|---|---|
| Mass | | APAP | APAPA | | | |
| Adsorb- | Vol- | Concen- | Concen- | grams | q (g APAP | % |
| ent | ume | tration | tration | APAP | adsorbed/g | APAP |
| (g) | (mL) | (g/L) | (g/L) | Adsorbed | adsorbent) | Adsorbed |
| 2 | 20 | 0.821 | 0.025 | 0.0159 | 0.0080 | 96.91 |
| 2 | 20 | 3.321 | 0.647 | 0.0535 | 0.0267 | 80.53 |
| 2 | 20 | 5.821 | 1.366 | 0.0891 | 0.0446 | 76.54 |
| 2 | 20 | 10.821 | 3.806 | 0.1403 | 0.0702 | 64.83 |
| 2 | 20 | 15.821 | 5.925 | 0.1979 | 0.0990 | 62.55 |

Methanol was used to elute the acetaminophen from the resins. Virtually all the acetaminophen was extracted into the methanol (Table 7). In this case, the resins also adsorbed some impurities and other compounds from the spent broth, which was co-eluted as well. The amount of impurities adsorbed was calculated to be ˜0.025 g. Adjusting the mass of final powder for this amount, the recovery yield from the methanol extraction and evaporation ranged from 45-85%, with an average of 68% (Table 8).

TABLE 7
Methanol Extraction of Acetaminophen from Resin, from Spent
Broth

| | Initial | | g APAP | | % APAP |
|---|---|---|---|---|---|
| | [APAP] | APAP g/L | extracted | g APAP in | extracted |
| Resin | (g/L) | in MeOH | in MeOH | resin | from resin |
| XAD4 | 0 | 2.095 | 0.021 | 0.015 | 144.47 |
| | 2.5 | 5.370 | 0.054 | 0.051 | 105.39 |
| | 5 | 8.231 | 0.082 | 0.081 | 101.93 |
| | 10 | 13.058 | 0.131 | 0.130 | 100.74 |
| | 15 | 18.673 | 0.187 | 0.183 | 102.03 |
| SP825L | 0 | 2.440 | 0.024 | 0.016 | 153.32 |
| | 2.5 | 5.393 | 0.054 | 0.053 | 100.82 |
| | 5 | 9.302 | 0.093 | 0.089 | 104.39 |
| | 10 | 14.404 | 0.144 | 0.140 | 102.67 |
| | 15 | 18.910 | 0.189 | 0.198 | 95.54 |

TABLE 8
Recovery of Acetaminophen by Methanol Extraction, from Spent
Broth

| | | | Mass | % | Mass of | |
|---|---|---|---|---|---|---|
| | | | of | APAP | Dry | |
| | | | Dry | Recov- | Powder | |
| | Initial | g APAP | Pow- | ery | (ad- | % APAP |
| | [APAP] | extracted | der | from | justed) | Recovery |
| Resin | (g/L) | in MeOH | (g) | MeOH | (g) | (Adjusted) |
| XAD4 | 0.821 | 0.021 | 0.039 | 186.12 | 0.014 | 66.81 |
| | 3.321 | 0.054 | 0.071 | 132.22 | 0.046 | 85.67 |
| | 5.821 | 0.082 | 0.077 | 93.55 | 0.052 | 63.18 |
| | 10.821 | 0.131 | 0.121 | 92.66 | 0.096 | 73.52 |
| | 15.821 | 0.187 | 0.146 | 78.19 | 0.121 | 64.80 |
| SP825L | 0.821 | 0.024 | 0.042 | 172.15 | 0.017 | 69.68 |
| | 3.321 | 0.054 | 0.067 | 124.24 | 0.042 | 77.88 |
| | 5.821 | 0.093 | 0.084 | 90.31 | 0.059 | 63.43 |
| | 10.821 | 0.144 | 0.09 | 62.48 | 0.065 | 45.13 |
| | 15.821 | 0.189 | 0.16 | 84.61 | 0.135 | 71.39 |

1. A non-naturally occurring microbial organism comprising at least three exogenous genes encoding acetaminophen pathway enzymes expressed in a sufficient amount to produce acetaminophen, wherein said acetaminophen pathway comprises (i) an enzyme that converts chorismic acid to p-aminobenzoic acid (ii) an enzyme that converts p-aminobenzoic acid to p-aminophenol and (iii) an enzyme that converts p-aminophenol to acetaminophen.

2. The non-naturally occurring microbial organism of claim 1 wherein said enzyme that converts chorismic acid to p-aminobenzoic acid is a two protein complex comprising ADC synthase and aminodeoxychorismate lyase; wherein said enzyme that converts p-aminobenzoic acid to p-aminophenol is 4-aminobenzoate 1-monoygenase and wherein said enzyme that converts p-aminophenol to acetaminophen is N-hydroxyarylamine O-acetyltransferase.

3. The non-naturally occurring microbial organism of claim 2 wherein said N-hydroxyarylamine O-acetyltransferase comprises SEQ ID NO: 4 or the active domain thereof.

4. The non-naturally occurring microbial organism of claim 1 wherein said enzyme that converts chorismic acid to p-aminobenzoic acid is a two protein complex comprising ADC synthase and aminodeoxychorismate lyase; wherein said enzyme that converts p-aminobenzoic acid to p-aminophenol is 4-aminobenzoate 1-monoygenase and wherein said enzyme that converts p-aminophenol to acetaminophen is arylamine N-acetyltransferase.

5. The non-naturally occurring microbial organism of claim 4 wherein said arylamine N-acetyltransferase comprises SEQ ID NO: 5 or the active domain thereof.

6.-9. (canceled)

10. The non-naturally occurring microbial organism of claim 2 wherein said ADC synthase comprises SEQ ID NO: 1 or the active domain thereof and said aminodeoxychorismate lyase comprises SEQ ID NO: 2 or the active domain thereof.

11. The non-naturally occurring microbial organism of claim 2 wherein said 4-aminobenzoate 1-monoygenase comprises SEQ ID NO: 3 or the active domain thereof.

12.-19. (canceled)

20. A method for producing acetaminophen comprising:
a. providing a fermentation media comprising carbon substrate; and
b. contacting said media with a recombinant yeast microorganism expressing an engineered acetaminophen biosynthetic pathway wherein said pathway comprises the following substrate to product conversions;
i. chorismic acid to p-aminobenzoic acid (PABA) (pathway step a);
ii. p-aminobenzoic acid to p-aminophenol (pathway step b);
iii. p-aminophenol to acetaminophen (pathway step c); and
c. culturing the yeast in conditions whereby acetaminophen is produced.

21. The method of claim 20 wherein
a) the substrate to product conversion of (i) is performed by a two protein complex comprising aminodeoxychorismate lyase and ADC synthase;
b) the substrate to product conversion of (ii) is performed by a 4-aminobenzoate 1-monoygenase enzyme; and
c) the substrate to product conversion of (iii) is performed by an enzyme selected from the group consisting of N-hydroxyarylamine 0-acetyltransferase and arylamine N-acetyltransferase.

22.-44. (canceled)

45. A method for purifying acetaminophen comprising
(a) filtering a liquid sample that comprises biologically derived acetaminophen with a reverse osmosis filter to produce a retentate;
(b) heating the retentate to 80° C. to evaporate liquid;
(c) cooling the remaining solution;
(d) filtering the solution;
(e) collecting the acetaminophen crystals; and
(f) drying the crystals to obtain purified acetaminophen.

46. (canceled)

47. The method of claim 45 further comprising re-solubilizing the acetaminophen crystals in distilled water and repeating process steps (a) thru (g).

48.-50. (canceled)

51. The non-naturally occurring microbial organism of claim 4, wherein said ADC synthase comprises SEQ ID NO: 1 or the active domain thereof and said aminodeoxychorismate lyase comprises SEQ ID NO: 2 or the active domain thereof.

52. The non-naturally occurring microbial organism of claim 4, wherein said 4-aminobenzoate 1-monoygenase comprises SEQ ID NO: 3 or the active domain thereof.


SEQUENCE LISTING (19 sequences)

SEQ ID NO: 1 - protein, 787 aa, Saccharomyces cerevisiae
MLSDTIDTKQQQQQLHVLFIDSYDSFTYNVVRLIEQQTDISPGVNAVHVTTVHSDTFQSMDQLLPLLPLFDAIVVGPGPGNPNNGAQDMGIISELFENANGKLDEVPILGICLGFQAMCLAQGADVSELNTIKHGQVYEMHLNDAARACGLFSGYPDTFKSTRYHSLHVNAEGIDTLLPLCTTEDENGILLMSAQTKNKPWFGVQYHPESCCSELGGLLVSNFLKLSFINNVKTGRWEKKKLNGEFSDILSRLDRTIDRDPIYKVKEKYPKGEDTTYVKQFEVSEDPKLTFEICNIIREEKFVMSSSVISENTGEWSIIALPNSASQVFTHYGAMKKTTVHYWQDSEISYTLLKKCLDGQDSDLPGSLEVIHEDKSQFWITLGKFMENKIIDNHREIPFIGGLVGILGYEIGQYIACGRCNDDENSLVPDAKLVFINNSIVINHKQGKLYCISLDNTFPVALEQSLRDSFVRKKNIKQSLSWPKYLPEEIDFIITMPDKLDYAKAFKKCQDYMHKGDSYEMCLTTQTKVVPSAVIEPWRIFQTLVQRNPAPFSSFFEFKDIIPRQDETPPVLCFLSTSPERFLKWDADTCELRPIKGTVKKGPQMNLAKATRILKTPKEFGENLMILDLIRNDLYELVPDVRVEEFMSVQEYATVYQLVSVVKAHGLTSASKKTRYSGIDVLKHSLPPGSMTGAPKKITVQLLQDKIESKLNKHVNGGARGVYSGVTGYWSVNSNGDWSVNIRCMYSYNGGTSWQLGAGGAITVLSTLDGELEEMYNKLESNLQIFM

SEQ ID NO: 2 - protein, 374 aa, Saccharomyces cerevisiae
MSLMDNWKTDMESYDEGGLVANPNFEVLATFRYDPGFARQSASKKEIFETPDPRLGLRDEDIRQQIINEDYSSYLRVREVNSGGDLLENIQHPDAWKHDCKTIVCQRVEDMLQVIYERFFLLDEQYQRIRIALSYFKIDFSTSLNDLLKLLVENLINCKEGNSEYHEKIQKMINERQCYKMRVLVSKTGDIRIEAIPMPMEPILKLTTDYDSVSTYFIKTMLNGFLIDSTINWDVVVSSEPLNASAFTSFKTTSRDHYARARVRMQTAINNLRGSEPTSSVSQCEILFSNKSGLLMEGSITNVAVIQKDPNGSKKYVTPRLATGCLCGTMRHYLLRLGLIEEGDIDIGSLTVGNEVLLFNGVMGCIKGTVKTKY

SEQ ID NO: 3 - protein, 460 aa, Agaricus bisporus
MSQQERTRVAIVGAGIVGLTLAIALNAFDKERKLAIDIYENASELAEIGAGINVWPRTLAIFKQIGVEDALIPLLDHIPDLEPRIIFGIRKGDEKNGYQVYDTMNNGGALRVHRAHLQNTLIQHLPLPGSKVTEINSICGFHLGHNLIDYSHHSSSGQGPLTLHFSDGKPSRTCDILVGADGIKSTLRHLFLPRLPNPEKYLNCYEPKWKGLLAYRGLVPKEKLEAVSPGHRALTHPGLMYSGKSAYAVVYPVSNGKFINVVAIVHDNPTNSTVWPGPWRMDVTQSEFFEVYKGWDEEVLDLIRCVDKPTKWALHALDHLDVYAKGRVFLMGDAAHAMLPHLGAGAHVGMEDAYILASLITHSSTPIWPSTQHVSEIANIYNTMRIPRAVSMSNSTDEAGYLCNLENPGLEEFKVGDHIPKELLIQTARTMEKKWAWTTTYADEDRIKAISLLEGPRAVL

SEQ ID NO: 4 - protein, 281 aa, Escherichia coli
MTPILNHYFARINWSGAAAVNIDTLRALHLKHNCTIPFENLDVLLPREIQLDNQSPEEKLVIARRGGYCFEQNGVFERVLRELGFNVRSLLGRVVLSNPPALPPRTHRLLLVELEEEKWIADVGFGGQTLTAPIRLVSDLVQTTPHGEYRLLQEGDDWVLQFNHHQHWQSMYRFDLCEQQQSDYVMGNFWSAHWPQSHFRHHLLMCRHLPDGGKLTLTNFHFTHYENGHAVEQRNLPDVASLYAVMQEQFGLGVDDAKHGFTVDELALVMAAFDTHPEAGK

SEQ ID NO: 5 - protein, 293 aa, Nocardia Farcinica
MSKPDDPAYHWNGAELDLDAYLARIGFAGERAPTLATLRELVYRHTTAIPFENLEAVLGRPVRLDLATLQDKLVHSRRGGYCYENAGLFAAALERLGFGVTGHTGRVTMGAGGLRPATHALLRVTTADDDRVWMCDVGFGRGPLRPYELRPQPDEFTLGDWRFRLERRTGELGTDLWVLHQFGRDGWVDRYTFTTAPQYRIDFEVGNHFVSTSPRSPFTTRPFLQRFHSDRHHVLDGLTLITERPDGSADIRALTPGELPEVINELFDIELPGPDLDALTTGSWLERVAAGTP

SEQ ID NO: 6 - protein, 193 aa, Lactococcus lactis
MKLLLIDNYDSFTYLLVQYFEELDCSVTVVNDQDKMSQKIRISPDFICENYDAITISPGPKTPKEAVFSRDVVQLYAGKIPMLGICLGQQVIAECFGGNVVLGERPMHGKISVIRHNCQGIFKGLPQNLKVARYHSLIVDKLPNDFEIDAQSEDGVIQAIHQPKLKLWALQFHPESLVTEYGHEMLNNFLKVV

SEQ ID NO: 7 - protein, 641 aa, Lactococcus lactis
MKEFIIKNTDIWKIFLKYYRSDEEIVFLHSSQATENEHYSILAHKPYKKVSKYKGQVFFNGEKKKFNFLDAVDLLKNEKVERPKNWPFYPELLGFVSYEQDPACFAAYDEVLLFDHRTKRLRVVQFEQTDGQYWLTESEEIEVDSEIEFDGQNGIGAVFIDQTRQEYIASIKRLQDYMKAGDIYVANLTQQFEIWSDQKPIDVFKKTRNQIPAPFSSFLQYPEWKMTQISSSVERFVSIHDGALISKPIKGTIARGEDVVTDRLQKEILSNSIKERTELLMVTDLLRNDIARISQPFSLSVPKFAEIETFSHVHQLVTSIKSRIKEDLTFSEFMTALFPGGSITGTPKKRAMEIIKEVEKQPRGIYTGMQGWLSREMDLDMNIVIRTLVHDGEHYQLGVGGGITLESEAEAEFSEILLKAKPFLDILGLKDVPSILFTTGLVKNGELLNLEGHVNRLKKQYHHPDLEEKLRKFAQNVTDGVLRVSTDGDSLNPEIRQLTHSNESYRVKLSSINDKPSPLSNFKLSGPDFQKVFRQEVLDVKKEGFQDILFHTDGLVSELSIGNFVAKKGNQYETPAKYALKGTFLDLFAKNHTLIYKDIAISDLKNYDCFYMTNAVRGLVEIKIDGISGSVAKFSKKSILV

SEQ ID NO: 8 - protein, 732 aa, Agaricus bisporus
MATVQPHVLLIDSYDSFAFNLAALVKKAIPDCTLHVIKNDSHTIQELLPSLSKFSAIVVGPGPGSPDIPTDIGVVCDIWKVANENLVPTFGVCLGQQSLGVENGATIHRLGTVKHGQVSVVKHTGTEIFAGLPDLKVVRYHSLHIRPKAGGEIEELAWSEDEDNGTIVMGVRHLSKPFWAVQYHPESILTDEGGLGIFRNFWKLASDWNTINRPHFRPQATLELSKSHVWPRVSKPYSANAANKTLGKVTFSTMEVPGLSVLRLCEFLGVNDESKRFVFLDSAAHPGQFSMIGCLNDDTAQFTHFVHENHIEESNAHSKVRHELGNKDVWTFFDEYREARKVVGGDRTVPFWGGFAGYLTYEAGFPPLGAGLKREDGHTAINHPDFNMVWVDRSIIVDIQNDKMWIQSLLPNDDAWIQETAQQISELPKGEIPVPTVAQGSSSVSFPDPEQYKTDVKRCQEFLFSGDSYELCLTGQTHITVPKEKTVPTWERYKILRGVNAAPHAGYLRLQPTTFLSCSPERFLSYSRSPGAVCELRPIKGTVRKMPHITREIAEEMLAGSRKEIAENLMIVDLIRHDLHNAVGENVVCSKFCGVEEYETLWQLVSVIEGSLPKDTPEDVDDSLGWEVLRRGLPPGSMTGAPKKRSVEILYNLEGFERGPYSGVFGYWCAGGGGDWSVTIRSVFLHDSEKNGDERWYVGAGGAITALSEPESELEETRVKLNGVLLGFGASS

SEQ ID NO: 9 - protein, 187 aa, Escherichia coli
MILLIDNYDSFTWNLYQYFCELGADVLVKRNDALTLADIDALKPQKIVISPGPCTPDEAGISLDVIRHYAGRLPILGVCLGHQAMAQAFGGKVVRAAKVMHGKTSPITHNGEGVFRGLANPLTVTRYHSLVVEPDSLPACFDVTAWSETREIMGIRHRQWDLEGVQFHPESILSEQGHQLLANFLHR

SEQ ID NO: 10 - protein, 453 aa, Escherichia coli
MKTLSPAVITLLWRQDAAEFYFSRLSHLPWAMLLHSGYADHPYSRFDIVVAEPICTLTTFGKETVVSESEKRTTTTDDPLQVLQQVLDRADIRPTHNEDLPFQGGALGLFGYDLGRRFESLPEIAEQDIVLPDMAVGIYDWALIVDHQRHTVSLLSHNDVNARRAWLESQQFSPQEDFTLTSDWQSNMTREQYGEKFRQVQEYLHSGDCYQVNLAQRFHATYSGDEWQAFLQLNQANRAPFSAFLRLEQGAILSLSPERFILCDNSEIQTRPIKGTLPRLPDPQEDSKQAVKLANSAKDRAENLMIVDLMRNDIGRVAVAGSVKVPELFVVEPFPAVHHLVSTITAQLPEQLHASDLLRAAFPGGSITGAPKVRAMEIIDELEPQRRNAWCGSIGYLSFCGNMDTSITIRTLTAINGQIFCSAGGGIVADSQEEAEYQETFDKVNRILKQLEK

SEQ ID NO: 11 - protein, 269 aa, Escherichia coli [sequence omitted]

SEQ ID NO: 12 - protein, 246 aa, Nitrosomonas europaea ATCC 19718
MATNTFKQQVDSIIQSRHLLQHPFYIAWTEGKLTREQLRHYAEQYFYNVLAEPTYLSAVHFNTPHFHNVENSGDISIRQEVLKNLIDEEHGEKNHPALWKAFAFALGADDASLTQADALPETENLVATFRDICINEPFYAGLAALHAFESQVPDIAAVKIDGLAKFYGMKDPDSYEFFSVHQTADIFHSQAEWAIIEKFADTPEKQAEVLAATRRACDALWKFLDGIHENYCANLICEEKTAATLH

SEQ ID NO: 13 - protein, 919 aa, Arabidopsis thaliana
MNMNFSFCSTSSELSYPSENVLRFSVASRLFSPKWKKSFISLPCRSKTTRKVLASSRYVPGKLEDLSVVKKSLPRREPVEKLGFVRTLLIDNYDSYTFNIYQALSTINGVPPVVIRNDEWTWEEAYHYLYEDVAFDNIVISPGPGSPMCPADIGICLRLLLECRDIPILGVCLGHQALGYVHGAHVVHAPEPVHGRLSGIEHDGNILFSDIPSGRNSDFKVVRYHSLIIDKESLPKELVPIAWTIYDDTGSFSEKNSCVPVNNTGSPLGNGSVIPVSEKLENRSHWPSSHVNGKQDRHILMGIMHSSFPHYGLQFHPESIATTYGSQLFKNFKDITVNYWSRCKSTSLRRRNINDTANMQVPDATQLLKELSRTRCTGNGSSYFGNPKSLFSAKTNGVDVFDMVDSSYPKPHTKLLRLKWKKHERLAHKVGGVRNIFMELFGKNRGNDTFWLDTSSSDKARGRFSFMGGKGGSLWKQLTFSLSDQSEVTSKHAGHLLIEDSQSSTEKQFLEEGFLDFLRKELSSISYDEKDFEELPFDFCGGYVGCIGYDIKVECGMPINRHKSNAPDACFFFADNVVAIDHQLDDVYILSLYEEGTAETSFLNDTEEKLISLMGLSTRKLEDQTLPVIDSSQSKTSFVPDKSREQYINDVQSCMKYIKDGESYELCLTTQNRRKIGNADPLGLYLHLRERNPAPYAAFLNFSNANLSLCSSSPERFLKLDRNGMLEAKPIKGTIARGSTPEEDEFLKLQLKLSEKNQAENLMIVDLLRNDLGRVCEPGSVHVPNLMDVESYTTVHTMVSTIRGLKKTDISPVECVRAAFPGGSMTGAPKLRSVEILDSLENCSRGLYSGSIGYFSYNGTFDLNIVIRTVIIHEDEASIGAGGAIVALSSPEDEFEEMILKTRAPANAVMEFCSDQRRQ

SEQ ID NO: 14 - protein, 456 aa, Marinobacterium jannaschii
MIRRHSLPYEEDSSTYFERVRSLGNPVYLDSCQPAALFGRFDIISAAPEALIRYHRGELTLQRATSGETLTDNPFDALRNLLDQYPQVAREKEFPFCGGLLGHFAYDLGRSLEAIPEQAADDIAFPEMRAGLYLWAVIVDHRLKQATLVAHPAISDSQWQLALNQLTAASTTAEDQFRLKKAFASNVDEAGYRHALNRIDDYIHAGDCYQVNFAQRFSAPYSGDPWQAYKQLRQHAPTPFAAYMESDDGEILSLSPERFLFTLENRVETKPIKGTRPRGKTPEEDQQQRDDLSKAIKDRAENLMIVDLLRNDLSKVCAFGSVRVPKLFDIESYANVHHLVTTVTGKLDQGQHPVDLLKHCFPGGSITGAPKIRAMEIIDELEPHRRSIYCGSIGYISLCGRMDTSITIRTLLCHDNKIHCWAGGGIVADSDIASEYAETFSKVNNLLGTLETTIQK

SEQ ID NO: 15 - protein, 723 aa, Streptomyces sp. FR-008
MRTLLVDNYDSFTYNLFHYLSRANGREPEVIRNDDPAWRPGLLDAFDNVVLSPGPGTPHRPADFGLCARIAEEGRLPVLGVCLGHQGMALAHGARVGRAPEPRHGRTSAVRHDGTGLFEGLPQPLEVVRYHSLAVTELPPELEATAWSEDGVLMALRHRTLPLWGVQFHPESIGTQDGHRLLANFRDLTERHGRTRPGGRAGHGTLPPPAPARETTATTGTPRRLRVIAESLPTRWDAEVAFDSLFRTGDHPFWLDSSRPGGELGQLSVMGDASGPLARTAKADVHAGTVTVRADGASSTVESAFLTWLENDLAGLRTEVPELPFAFALGWVGCLGYELKAECDGDAAHRSPDPDAVLVFADRALVLDHRTRTTYLLALVEDDAEAEARAWLAAASATLEAIAGREPEPCPEAPVCTTGPVELRHDRDGYLKLIDVCQQEIAAGETYEVCLTNMAEADTDLTPWAAYRALRRVSPAPFAAFLDFGPMAVLSSSPERFLRIDRHGRMESKPIKGTRPRGATPQEDAALVRALATCEKDRAENLMIVDLVRHDLGRCAEVGSVVADPVFQVETYATVHQLVSTVTARLREDSSPVAAVRAAFPGGSMTGAPKIRTMQIIDRLEGGPRGVYSGAIGYFSLTGAVDLSIVIRTVVLSGGRLRYGVGGAVIALSDPADEFEETAVKAAPLLRLLDTAFPGREAPGKDLDGEPDDGTDAGAPKDLVLPG

SEQ ID NO: 16 - protein, 257 aa, Streptomyces sp. FR-008
MIELDGEPAGPEALASLALTNYGHFTTLLVENGRVRGLDLHLERLIRDCRTLFDAALDPDRVRKLARRAAPTDGRATVRVTVFDPALNLGNIAADARPGILVTSRPAPDKPPGPLRVRSVVHRRDLPEVKSVGLCPTLRLRRQAQRAGYDDVLFTGPDGDILEGGTWNVGLVRDGEVVWPGGEVLAGTTRQLLRRATDGPTELVGLADLDSVEAVFATNAAVGVRPVTGIDDREFPAAHHSVTRLAEIYQALPGSPL

SEQ ID NO: 17 - protein, 669 aa, Streptomyces venezuelae  ATCC 10712
MRTLLIDNYDSFTQNLFQYIGEATGQPPVVPNDADWSRLPLEDFDAIVVSPGPGSPDRERDFGISRRAITDSGLPVLGVCLGHQGIAQLSAEPMHGRVSEVRHTGEDVFRGLPSPFTAVRYHSLAATDLPDELEPLAWSDDGVVMGLRHREKPLMGVQFPPESIGSDFGREIMANFRDLALAHHRARRDAADWGYELHVRRVDVLPDAEEVRRAACPAEGATFWLDSSSVLEGASPFSFLGDDRGPLAEYLTYRVADGVVSVRGSDGTTTRDAATLFSYLEEQLEPPAGPVAPDLPFEFNLGYVGYLGYELKAETTGDPAVPAPHPDAAFLFADRAIALDHQEGCCYLLALDRRGHDDGARAWLRETAETLTGLAVRVRPRPTPAMVFGVPEAAAGFGPLARARHDKDASALRNGESYEICLTNMVTAPTEATALPLYSALRRISPVPSGALLEFPELSVLSASPERFLTIGADGGVESKPIKGTRPRGAPAEEDERLRADLAGREKDRAENLMIVDLVRNDLNSVCAIGSVHVPRLFEVGDLAPVHQLVSTIRGRLRPGTSTAACVRAAFPGGSMTGAPKKRPMEIIDRLEEGPRGVLPGALGWFALSGAADLSIVIRTIVLADGRAEFGVGGAIVSLSDQEEEFRQTVVKARAMVTALDGSAVAGAR

SEQ ID NO: 18 - protein, 253 aa, Streptomyces venezuelae  ATCC 10712
MTTAPPHIEIDGDPAADPALLATLMSGYGHFTAMQVRDGRVKGLDLHLARLDGATRELFGQELPGRRVRALIAGALKTSGQRDASVRVYVYEGPRIAVTVAPPTPDGPGAPRRLTTVEYWRPAAHIKHLGGFGQSYHLEAARRAGYDEALLTSPYGEIAEGAITNIGFWDGSSLVWPSAPCLDGIAMLLLRSRLESVSRPVTLADLPGYRAAFTTNSRGISPVTAIDDVTFAVDEELMGRVYSAYDSVEWDAL

SEQ ID NO: 19 - protein, 883 aa, Trichoderma reesei RUT C-30
MPLLESQSAAGLSASTPAEAAQDGRRPARRILFLDAYDSFTNNIVSLLKEALGDDVLVHVLHMDLKTLHADPGPDWTPEQFLARLPAFDAVVCGPGPGSPLCEADVGAFRLLWRLRDEAAVPVLGICLGFQSLVAHFGGGIRRLRRGLHGMVRDIEHRSGDIFAGVPPFRATLYHSLCADVGQDDVSRDAWETDMWLPPKGAPDLVPLAWTMEESEDAEAEPERILMGVRHARLPFWGVQYHPESVCTDAAAQGVLKNWFSQALKWNEVTGRRVRPVDFEHIFDSLAPATIVERRLAAQNHIATQKWWRDMQSGLAASRAAPVYTCRRIKLPDGVDAADVAELLGVGGVDSIMLDSSSTVNGDPLARSSVIALEVDQALRFEYHVGDNWVTLRQPGAGGQQETCQRIEIDRSDEHGAYDVWNVISEYWQQRKIAEEDGQEPAFKGGFMGFVTYEMGLGTLTPKSVSDSRGHHRPDLCFAWISKSLVLDHQAGVAYVQALTVPDSDAQSWIDEIVAKLQSSRAWQQPGYGADEYRAIAAKKQANEALLKSIQEPSGRLRFNTPQPNKYEDDVRQCQDAIAEGQSYELCLTAQTKMIRPRGDDVPHHLRPSSDGPSNNEAASPAWPSGEEASSHGTPWQIYRTLRARQPAPYGSFIRLGGATILSSSPERFLGHDAKGLCSMRPMKGTVRKSEAVSTLAQAEALLHVPKEEAENLMIVDLVRHDLYGVCGARNVTVPDLLKVEDYSSVFQMITVVNGQLPSSSRAAPNGLDVLAAALPPGSMTGAPKKRSCEILRALEPEERSIYSGVVGFFDARGHGGWSVTIRTMFRWDDEEAPPEGPGETRPREVWRIGAGGAVTILSTPEGETEEMFIKLCGPLGVFKDAA